(19) United States
(12) Patent Application Publication
(10) Pub. No.: US 2023/0148871 A1
(43) Pub. Date: May 18, 2023

(54) DETECTING CORE BODY TEMPERATURE BASED ON SENSED EXHALED BREATH IN A PPE RESPIRATOR DEVICE

(71) Applicant: 3M INNOVATIVE PROPERTIES COMPANY, St. Paul, MN (US)

(72) Inventors: William Bedingham, Woodbury, MN (US); Jordan J.W. Craig, White Bear Lake, MN (US); Andrew W. Long, Woodbury, MN (US); Richard J. Sabacinski, Charlotte, NC (US); John R. Stark, St. Paul, MN (US); Daniel B. Taylor, White Bear Lake, MN (US); Caroline M. Ylitalo, Stillwater, MN (US); Chin-Yee Ng, Oakdale, MN (US); Christopher M. Brown, Cottage Grove, MN (US); Eric H. Tsai, St. Paul, MN (US); Glenn E. Casner, Woodbury, MN (US); Jeremy W. Nueman, Stillwater, MN (US); Jia Hu, Mounds View, MN (US); Mark G. Mathews, Woodbury, MN (US); Maioding Dai, Woodbury, MN (US); Perry S. Dotterman, Maplewood, MN (US); Travis W. Rasmussen, Rosemount, MN (US); William K. Preska, Woodbury, MN (US)

(21) Appl. No.: 17/996,790

(22) PCT Filed: Apr. 21, 2021
(86) PCT No.: PCT/IB2021/053294

§ 371 Date: Oct. 21, 2022

Related U.S. Application Data
(60) Provisional application No. 63/018,138, filed on Apr. 30, 2020.

(51) Int. Cl. A61B 5/01; A61B 5/00; A62B 18/02

(52) U.S. Cl. CPC A61B 5/01; A61B 5/6803; A61B 5/746; A62B 18/02; A61B 2560/0252

(57) ABSTRACT

Example systems are described in which a personal protective equipment (PPE) respirator device includes one or more sensor arranged within the PPE to detect exhalation breath temperature of a user, and a computing device configured to generate, based on the detected exhalation breath temperature, a metric indicative of core body temperature of the user wearing the PPE.

### TECHNICAL FIELD

The present disclosure relates to personal protective equipment (PPE) and more specifically, PPE respirator devices.

### BACKGROUND

Many work environments present hazards that may expose workers to a safety event, such as a fall, breathing contaminated air, or temperature related injuries (e.g., heat stroke, heat exhaustion, hypothermia, frostbite, or other injury related to temperature). In many work environments, workers may utilize personal protective equipment (PPE) to help mitigate the risk of a hazardous safety event. Often, a worker may not recognize an impending hazardous safety event until the environment becomes too dangerous or until the worker's health has experienced significant deterioration.

PPE respirator devices come in many different styles and designs. Examples include a filtered face piece respirator (FIR), a negative pressure respirator, a powered air purifying respirator (PAPR), a self-contained breathing apparatus (SCBA) that includes a respirator, and many other types. In some cases, PPE respirator devices may comprise an integrated part of other safety equipment. For example, respirator devices may also be used within SCBA equipment, welding masks, flight helmets, underwater breathing devices, firefighter helmets or masks, military equipment, and/or a wide variety of other devices or equipment.

Detection of core body temperature can be very useful for monitoring workers in extreme environments and rigorous working conditions. Unfortunately, techniques for detecting core body temperature in work settings are very invasive. For example, some conventional techniques for detecting core body temperature use temperature sensors implanted or inserted into the body, such as by swallowing a pill-like sensor. Other techniques may use rectal implants for core body temperature detection.

### SUMMARY

In general, this disclosure describes example systems in which a personal protective equipment (PPE) respirator device that includes one or more sensors arranged within the PPE to detect exhalation breath temperature of a user, and a computing device configured to generate, based on the detected exhalation breath temperature, a metric indicative of core body temperature of the user wearing the PPE. As explained herein, exhalation breath temperature may be used as a proxy or correlated metric for detecting problems with core body temperature. Although exhalation breath temperature itself may not always accurately indicate the exact core body temperature, techniques described herein may be used to estimate core body temperature of a user of a PPE respirator based on detected exhalation breath temperature. In particular, exhalation breath temperature may be sensed and used to define a metric indicative of core body temperature or to detect notable changes to core body temperature of a user wearing one or more PPEs. By integrating a temperature sensor into a PPE respirator device at a proper location, as described herein, to effectively measure exhalation breath temperature (and possibly other quantities like air humidity, breath rate, or physiological parameters of the user), the PPE respirator device may be useful for helping to monitor core body temperature of a worker and help promote worker safety.

In one example, this disclosure describes a PPE system comprising a PPE respirator device including a sensor arranged to detect exhalation breath temperature of a user, and a computing device configured to generate a metric indicative of core body temperature of the user based on the detected exhalation breath temperature.

In another example, this disclosure describes a PPE respirator device comprising a facepiece designed to fit over a nose and a mouth of a user, the facepiece defining a sealable space formed around the nose and the mouth of the user for delivering air to the user, wherein the facepiece includes an inhalation valve and an exhalation valve, a first sensor positioned proximate to the exhalation valve and configured to detect exhalation breath temperature, and a second sensor positioned proximate to the inhalation valve and configured to detect inhalation breath temperature.

In another example, this disclosure describes a method that includes detecting exhalation breath temperature inside a PPE respirator device and generating a metric indicative of core body temperature based at least in part on the detected exhalation breath temperature.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a block diagram illustrating an example system in which a negative pressure re-usable respirator includes an exhalation breath sensor and is in communication with a personal protection equipment management system, in accordance with various techniques of this disclosure.

FIG. 2 is a block diagram illustrating, in detail, an example operating perspective of the personal protection equipment management system shown in FIG. 1.

FIG. 3 is a conceptual diagram illustrating an example negative pressure re-usable respirator device that includes breath sensors, in accordance with various techniques of this disclosure.

FIGS. 4 and 5 are block diagrams illustrating two example systems comprising a PPE respirator device with breath sensors in communication with a computing device.

FIG. 6 is a perspective view of an example negative pressure re-usable respirator device that includes breath sensors.

FIG. 7 is a perspective view of an example prototype of a negative pressure re-usable respirator device that includes breath sensors.

FIG. 8 is a perspective view inside of a molded cap of prototype of FIG. 7, whereby the molded cap houses a circuit that may form part of a respirator device.

FIG. 9 is a perspective disassembled view of the prototype of FIG. 7 showing breath sensors located in proximity to inhalation and exhalation valves of the negative pressure re-usable respirator device.

FIGS. 10 and 11 are flow diagrams consistent with techniques of this disclosure.

It is to be understood that the embodiments may be utilized and structural changes may be made without departing from the scope of the invention. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

This disclosure describes various examples in which one or more breath sensors are integrated into a personal protection equipment (PPE) respirator device, such as a full-face respirator device, a half-face respirator device, a filtered face piece respirator (FFR) a negative pressure reusable respirator system, a powered air purifying respirator (PAPR), a filtered face piece respirator (FFR), a respirator that forms part of a self-contained breathing apparatus (SCBA), a respirator that forms part of a safety helmet or mask, a respirator that forms part of firefighter safety equipment, a respirator that forms part of military equipment, or another type of respirator or respirator system. In still other cases, the PPE respirator device may comprise a medical respirator device such as a continuous positive airway pressure (CPAP) device or oxygen mask commonly used in hospital and care settings.

As further described herein, an example system may comprise a PPE respirator device that includes a sensor arranged to detect exhalation breath temperature of a user, and a computing device configured to generate a metric indicative of core body temperature of the user based on the detected exhalation breath temperature. In various examples, the computing device may be integrated with the sensor, may be electrically coupled to the sensor within the PPE respirator device, may be a local computing device associated with the user of the PPE respirator device and in wireless communication with the sensor, or may be totally separate from the PPE respirator device an part of a larger work system environment. In these and other examples, the PPE respirator device with an integrated breath sensor may be used in isolation with a PPE respirator device, or may be used within a larger “connected” work environment whereby workers are monitored by other people (e.g., supervisors) in the connected work environment.

In various examples, additional sensors may also be used to help improve calculation of the metric indicative of core body temperature, such as sensors configured to detect inhalation breath temperature, sensors arranged to detect ambient conditions, humidity sensors, and/or other types of sensors. Such additional sensors may provide information that, in combination with information about exhalation breath temperature, may be used to define a metric indicative of core body temperature or changes thereto.

Exhalation breath temperature may be used as a proxy or correlated metric for detecting problems with core body temperature. Although exhalation breath temperature may not itself accurately indicate the exact core body temperature, exhalation breath temperature may correlate with core body temperature. Therefore, exhalation breath temperature may be sensed and used to define a metric indicative of core body temperature or to detect notable changes to core body temperature. By integrating a temperature sensor into a PPE respirator device at a proper location to effectively measure exhalation breath temperature (and possibly other quantities like humidity, breath rate, or physiological parameters of the user), the PPE respirator device may be useful for helping to monitor core body temperature of a worker and help promote worker safety.

The breath sensor(s) may allow for detection of exhalation breath temperature of a user of the PPE respirator device. Furthermore, the breath sensor(s) may be used to detect changes in core body temperature, such as the time rate of change in core body temperature or acceleration of the rate of change of a user's core body temperature. Such changes or acceleration of changes in core body temperature may correlate with changes or acceleration of changes in exhalation breath temperature. Environmental factors, such as ambient temperature of the working environment, humidity, and air quality, may also affect the user's core body temperature, so in some cases, these addition factors may be measured and used (in addition to exhalation breath temperature) in defining the metric indicative of core body temperature.

A variety of different types of PPE respirator devices are commonly used in harsh environmental settings or other situations where the monitoring of core body temperature is desirable. By integrating one or more breath sensors (and possibly other sensors) into PPE respirator device, the PPE respirator device can be used to help track or monitor a metric indicative of core body temperature. Most conventional techniques for detecting core body temperature are very invasive. The techniques and structure described in this disclosure may help to promote user comfort and help to avoid more invasive techniques or devices that might otherwise be used to monitor core body temperature.

In some examples, the breath sensor is also electrically coupled to a circuit that, like the exhalation breath sensor, is integrated into the PPE respirator device in manner that does not interfere with its normal respiratory operation. This can help provide environmental protection for the circuit and provide a useful and ergonomic design. The circuit, for example, may include wireless communication capabilities so that sensor data collected by the exhalation breath sensor can be sent to one or more external computing devices, such as a computing device associated with the user wearing the PPE device, or a computing device associated with other users that monitor the user wearing the PPE device. The computing device may process sensed data to determine a metric indicative of core body temperature. The computing device may generate one or more alerts when the metric indicates one or more possible issues related to core body temperature. In other examples, the computing device and the sensor may be integrated into a package that is not wirelessly connected to anything, which may be useful in environments where wireless communication is forbidden or impossible. In still other examples, the computing device and the sensor may be geographically separated, and sensor data from the exhalation breath sensor may be sent from the PPE respirator device to the computing device via network.

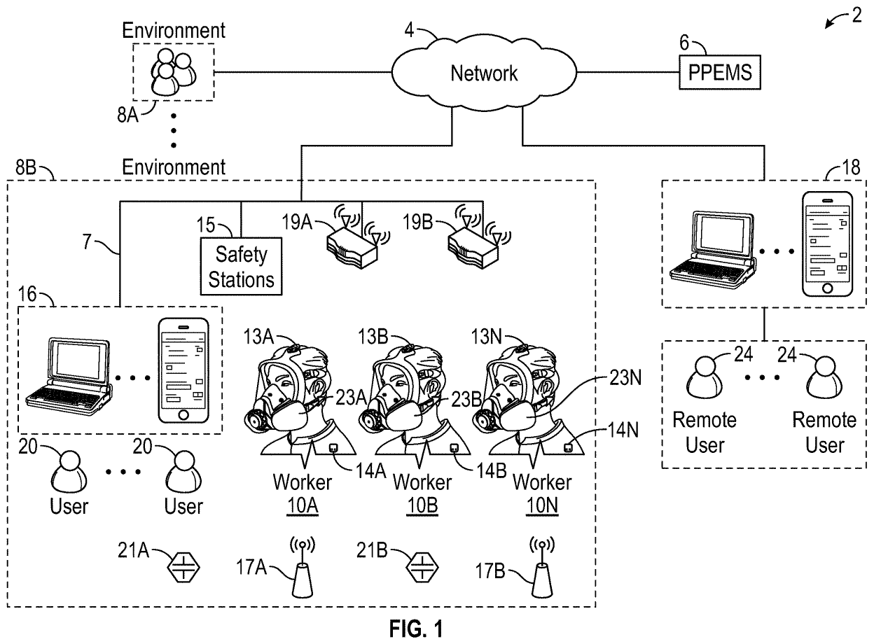

FIG. 1 is a block diagram illustrating an example system 2 in which negative pressure respirators 13 include one or more sensors positioned within or around a facepiece and configured to detect exhalation breath temperature of a user. As described herein, the detected exhalation breath temperature may be processed by a computing device in order to generate a metric indicative of core body temperature. Additional sensors may also be used to help improve or refine the metric indicative of core body temperature. Negative pressure re-usable respirators 13A-13N are shown merely for illustrative purposes and may generally represent any type of PPE respirator device.

According to this disclosure, negative pressure re-usable respirators 13A-13N (collectively, negative pressure re-usable respirators 13) include one or more sensors to detect conditions of the respective negative pressure re-usable respirators 13. More specifically, one or more of respirators 13 may include an exhalation breath sensor that is positioned inside or around a facepiece of the respirator (e.g., near an exhalation valve of the facepiece). In addition, respirators 13 may also include other sensors for detecting other parameters associated with the worker or the working conditions.

One or more computing devices (e.g., any of personal protective equipment management system (PPEMS) 6, hubs 14, among others) utilize the sensor data from the sensors of the negative pressure re-usable respirators 13 to detect or predict safety events associated with negative pressure re-usable respirators 13. As used in this disclosure, safety events may be defined based on sensed physiological conditions of the user, such as core body temperature, sudden changes in order body temperature, acceleration in such changes, or deviations from expected core body temperature. However, sensors may also be used to identify other safety events, such as saturation or loading of a contaminant capture device of a negative pressure re-usable respirator (e.g., blockage of a particulate filter), exhaustion of a contaminant capture device (e.g., break though of a chemical cartridge), incompatibility between the hazards a contaminant capture device is configured to protect against and hazards within a work environment, insufficient seal between the respirator and the worker's face, among others.

According to techniques of this disclosure, the one or more computing devices, such as PPEMS 6, monitor data provided by the exhalation breath sensor (or other sensors) within PPEs 13 in order to detect and/or predict safety events and alert workers of such safety events. In some examples, PPEMS 6 also monitor usage of contaminant capture devices 23A-23N of negative pressure re-usable respirators 13 and determine whether the contaminant capture device (e.g., a particulate filter) is due for replacement. As another example, PPEMS 6 may determine whether the air within a sealable space defined by (e.g., formed between) a worker's face and a respective negative pressure re-usable respirator 13 is sealed from air within the work environment (e.g., air exterior to the respirator). In some instances, PPEMS 6 determines whether the contaminant capture device utilized by a particular worker is working properly with a useful filter to protect the worker from hazards within the work environment. Furthermore, PPEMS 6 may determine and monitor physiological parameters of the user, such as heart rate, core body temperature, respiratory rate, blood pressure, blood oxygen saturation levels, or other physiological parameters, in order to promote user safety.

As shown in the example of FIG. 1, system 2 represents a computing environment in which computing device(s) within a plurality of physical environments 8A, 8B (collectively, environments 8) electronically communicate with PPEMS 6 via one or more computer networks 4. Each of physical environment 8 represents a physical environment, such as a work environment, in which one or more individuals, such as workers 10, utilize personal protective equipment 13 while engaging in tasks or activities within the respective environment. Example environments 8 include construction sites, mining sites, manufacturing sites, among others.

In the example of FIG. 1, environment 8A is shown as generally as having workers 10, while environment 8B is shown in expanded form to provide a more detailed example. In this example, a plurality of workers 10A-10N are shown as utilizing personal protective equipment (PPE), such as negative pressure re-usable respirators 13. As used throughout this disclosure, negative pressure re-usable respirators 13 include any re-usable respirator in which the air pressure inside the facepiece is less than the ambient air pressure (e.g., the pressure of the air outside the respirator) during inhalation.

Although respirators 13 in the example of FIG. 1 are illustrated as negative-pressure re-usable respirators, the techniques described herein apply to other types of respirators, such as positive pressure re-usable respirators, disposable respirators, or powered-air purifying respirators. As used throughout this disclosure, a positive pressure respirator includes any respirator in which the air pressure inside the facepiece is greater than the ambient air pressure. Negative pressure re-usable respirators 13 include a facepiece (e.g., a full facepiece, or a half facepiece) configured to cover at least a worker's nose and mouth. For example, a half facepiece may cover a worker's nose and mouth and a full facepiece may cover a worker's eyes, nose, and mouth. Negative pressure re-usable respirators 13 may fully or partially (e.g., 75%) cover a worker's head. Negative pressure re-usable respirators 13 may include a head harness (e.g., an elastic strap) that secures negative pressure re-usable respirators 13 to the back of the worker's head. Monitoring core body temperature of a user ay be useful in any such devices or other similar devices.

In some examples, negative pressure re-usable respirators 13 are configured to receive contaminant capture devices 23A-23N (collectively, contaminant capture devices 23). Contaminant capture devices 23 are configured to remove contaminants from air as air is drawn through the contaminant capture device (e.g., when a worker wearing a reusable respirator inhales). Contaminant capture devices 23 may include particulate filters, chemical cartridges, or combination particulate filters/chemical cartridges. As used throughout this disclosure, particulate filters are configured to protect a worker from particulates (e.g., dust, mists, fumes, smoke, mold, bacteria, or other undesirable particulates). Particulate filters capture particulates through impaction, interception, and/or diffusion. As used throughout this disclosure, chemical cartridges are configured to protect a worker from gases or vapors. Chemical cartridges may include sorbent materials (e.g., activated carbon) that react with a gas or vapor to capture the gas or vapor and remove the gas or vapor from air breathed by a worker. For instance, chemical cartridges may capture organic vapors, acid gasses, ammonia, methylamine, formaldehyde, mercury vapor, chlorine gas, among others.

Contaminant capture devices 23 may be removable. In other words, a worker may remove a contaminant capture device from a negative pressure re-usable respirator 13 (e.g., upon the contaminant capture device reaching the end of its expected lifespan) and install a different (e.g., unused, new) contaminant capture device to the respirator. In some examples, the particulate filters or chemical cartridges have a limited service life. In some examples, when a chemical cartridge is exhausted (e.g., captures a threshold amount of gas or vapors), gases or vapors may pass through the chemical cartridge to the worker (which is called “breakthrough”). In some examples, as particulate filters become saturated with a contaminant, the filter becomes harder to pull air through, thus making the worker inhale deeper to breathe.

In the example of FIG. 1, each of negative pressure re-usable respirators 13 include, in some examples, embedded sensors or monitoring devices and processing electronics configured to capture data in real-time as a user (e.g., worker) engages in activities while utilizing (e.g., wearing) the respirator.

For example, as described in detail herein, one or more of respirators 13 may comprise a facepiece designed to fit over a nose and a mouth of a user, the facepiece defining a sealable space formed around the nose and the mouth of the user for delivering air to the user, wherein the facepiece includes an inhalation valve and an exhalation valve. A sensor may be positioned proximate to the exhalation valve and configured to detect exhalation breath temperature. In some cases, a first sensor may be positioned proximate to the inhalation valve and configured to detect inhalation breath temperature, and a second sensor may be positioned proximate to the exhalation valve and configured to detect exhalation breath temperature. For example, the first sensor may be positioned inside the sealable space proximate to the exhalation valve of the facepiece, and the second sensor may be positioned inside the sealable space proximate to the inhalation valve of the facepiece. In another example, the first sensor may be positioned outside the sealable space proximate to the exhalation valve of the facepiece, and the second sensor may be positioned outside the sealable space proximate to the inhalation valve of the facepiece. In still another example, the first sensor may be positioned inside the sealable space proximate to the exhalation valve of the facepiece and the second sensor may be positioned outside the sealable space proximate to the inhalation valve of the facepiece. These and other configurations may be used.

In addition, negative pressure re-usable respirators 13 may also include a number of additional sensors for sensing operational characteristics of the respirators 13. For example, each of respirators 13 may include an air pressure sensor configured to detect the air pressure in the cavity formed between the respirator and the worker's face, which detect the air pressure within the cavity as the worker 10 breathes (e.g., inhales and exhales). In other words, the air pressure sensors detect the air pressure within the sealed space (also referred to as a cavity, or respirator cavity) formed by a face of the worker and the negative pressure reusable respirator. In addition, each of negative pressure re-usable respirators 13 may include one or more output devices for outputting data that is indicative of operation of negative pressure re-usable respirator 13 and/or generating and outputting communications to the respective worker 10. For example, negative pressure re-usable respirators 13 may include one or more devices to generate audible feedback (e.g., one or more speakers), visual feedback (e.g., one or more displays, light emitting diodes (LEDs) or the like), or tactile feedback (e.g., a device that vibrates or provides other haptic feedback). More specific details on a breath sensors of respirators 13, as well as techniques for determining an indication of core body temperature based on exhalation breath temperature are provided in greater detail below.

Each of negative pressure re-usable respirators 13 is configured to communicate data, such as sensed motions, events and conditions, via wireless communications, such as via 802.11 WiFi® protocols, Bluetooth® protocol or the like. Negative pressure re-usable respirators 13 may, for example, communicate directly with a wireless access point 19. As another example, each worker 10 may be equipped with a respective one of wearable communication hubs 14A-14M that enable and facilitate communication between negative pressure re-usable respirators 13 and PPEMS 6. For example, negative pressure re-usable respirators 13 as well as other PPEs (such as fall protection equipment, hearing protection, hardhats, or other equipment) for the respective worker 10 may communicate with a respective communication hub 14 via Bluetooth or other short-range protocol, and the communication hubs may communicate with PPEMs 6 via wireless communications processed by wireless access points 19. Although shown as wearable devices, hubs 14 may be implemented as stand-alone devices deployed within environment 8B. In some examples, hubs 14 may be articles of PPE.

In general, each of environments 8 include computing facilities (e.g., a local area network) by which sensing stations 21, beacons 17, and/or negative pressure re-usable respirators 13 are able to communicate with PPEMS 6. For examples, environments 8 may be configured with wireless technology, such as 802.11 wireless networks, 802.15 ZigBee networks, and the like. In the example of FIG. 1, environment 8B includes a local network 7 that provides a packet-based transport medium for communicating with PPEMS 6 via network 4. Environment 8B may include wireless access point 19 to provide support for wireless communications. In some examples, environment 8B may include a plurality of wireless access points 19 that may be geographically distributed throughout the environment to provide support for wireless communications throughout the work environment.

In some examples, each worker 10 may be equipped with a respective one of wearable communication hubs 14A-14N that enable and facilitate wireless communication between PPEMS 6 and sensing stations 21, beacons 17, and/or negative pressure re-usable respirators 13. For example, sensing stations 21, beacons 17, and/or negative pressure re-usable respirators 13 may communicate with a respective communication hub 14 via wireless communication (e.g., Bluetooth® or other short-range protocol), and the communication hubs may communicate with PPEMS 6 via wireless communications processed by wireless access point 19. Although shown as wearable devices, hubs 14 may be implemented as stand-alone devices deployed within environment 8B.

In general, each of hubs 14 is programmable via PPEMS 6 so that local alert rules may be installed and executed without requiring a connection to the cloud. As such, each of hubs 14 provides a relay of streams of data from sensing stations 21, beacons 17, and/or negative pressure re-usable respirators 13, and provides a local computing environment for localized alerting based on streams of events in the event communication with PPEMS 6 is lost.

As shown in the example of FIG. 1, an environment, such as environment 8B, may also contain one or more wireless-enabled beacons, such as beacons 17A-17B, that provide accurate location data within the work environment. For example, beacons 17A-17B may be GPS-enabled such that a controller within the respective beacon may be able to precisely determine the position of the respective beacon. Based on wireless communications with one or more of beacons 17, a given negative pressure re-usable respirator 13 or communication hub 14 worn by a worker 10 is configured to determine the location of the worker within environment 8B. In this way, event data reported to PPEMS 6 may be stamped with positional data to aid analysis, reporting and analytics performed by PPEMS 6.

In addition, an environment, such as environment 8B, may also include one or more wireless-enabled sensing stations, such as sensing stations 21A, 21B. Each sensing station 21 includes one or more sensors and a controller configured to output data indicative of sensed environmental conditions. Moreover, sensing stations 21 may be positioned within respective geographic regions of environment 8B or otherwise interact with beacons 17 to determine respective positions and include such positional data when reporting environmental data to PPEMS 6. As such, PPEMS 6 may be configured to correlate the sensed environmental conditions with the particular regions and, therefore, may utilize the captured environmental data when processing event data received from negative pressure re-usable respirators 13, or sensing stations 21. For example, PPEMS 6 may utilize the environmental data to aid generating alerts or other instructions for negative pressure re-usable respirators 13 and for performing predictive analytics, such as determining any correlations between certain environmental conditions (e.g., heat, humidity, visibility) with abnormal worker behavior or increased safety events. As such, PPEMS 6 may utilize current environmental conditions to aid prediction and avoidance of imminent safety events. Example environmental conditions that may be sensed by sensing stations 21 include but are not limited to temperature, humidity, presence of gas, pressure, visibility, wind, or other conditions. Safety events may refer to heat related illness or injury, cardiac related illness or injury, respiratory related illness or injury, or eye or hearing related injury or illness. Worker problems may be identified based on physiological changes in the worker such as core body temperature, heart rate, respiratory rate, which may be tracked by a breath sensor (or other sensors) as described herein.

In example implementations, an environment, such as environment 8B, may also include one or more safety stations 15 distributed throughout the environment. Safety stations 15 may allow one of workers 10 to check out negative pressure re-usable respirators 13 and/or other safety equipment, verify that safety equipment is appropriate for a particular one of environments 8, and/or exchange data. Safety stations 15 may enable workers 10 to send and receive data from sensing stations 21, and/or beacons 17. For example, safety stations 15 may transmit alert rules, software updates, or firmware updates to negative pressure re-usable respirators 13 or other equipment, such as sensing stations 21, and/or beacons 17. Safety stations 15 may also receive data cached on negative pressure re-usable respirators 13, hubs 14, sensing stations 21, beacons 17, and/or other safety equipment. That is, while equipment such as sensing stations 21, beacons 17, negative pressure re-usable respirators 13, and/or data hubs 14 may typically transmit data via network 4 in real time or near real time, such equipment may not have connectivity to network 4 in some instances, situations, or conditions. In such cases, sensing stations 21, beacons 17, negative pressure re-usable respirators 13, and/or data hubs 14 may store data locally and transmit the data to safety stations 15 upon regaining connectivity to network 4. Safety stations 15 may then obtain the data from sensing stations 21, beacons 17, negative pressure re-usable respirators 13, and/or data hubs 14.

In addition, each of environments 8 may include computing facilities that provide an operating environment for end-user computing devices 16 for interacting with PPEMS 6 via network 4. For example, each of environments 8 typically includes one or more safety managers responsible for overseeing safety compliance within the environment. In general, each user 20 interacts with computing devices 16 to access PPEMS 6. Each of environments 8 may include systems. Similarly, remote users may use computing devices 18 to interact with PPEMS 6 via network 4. For purposes of example, the end-user computing devices 16 may be laptops, desktop computers, mobile devices such as tablets or so-called smart phones and the like.

Users 20, 24 interact with PPEMS 6 to control and actively manage many aspects of safely equipment utilized by workers 10, such as accessing and viewing usage records, analytics and reporting. In addition, users 20, 24 may review physiological data acquired and stored by PPEMS 6, where the data may include data specifying physiological metrics associated with a user, such as core body temperature, heart rate, respiratory rate, blood oxygen levels, baselines, trends, or changes in such physiological metrics. Other sensed data that may be acquired and stored by PPEMS 6 may include such things as starting and ending times over a time duration (e.g., a day, a week, or another periodic time interval), data collected during particular events, such as pulling a respirator away from the worker's face (e.g., such that the cavity formed by the worker's face and the respirator is not sealed, which may expose the worker to breathing hazards, without necessarily removing the respirator from the worker 10), removal of a negative pressure re-usable respirator 13 from a worker 10, changes to operating parameters of a negative pressure re-usable respirator 13, status changes to components of negative pressure re-usable respirators 13 (e.g., a low battery event), motion of workers 10, detected impacts to negative pressure re-usable respirators 13 or hubs 14, sensed data acquired from the user, environment data, and the like. In addition, users 20, 24 may interact with PPEMS 6 to perform asset tracking and to schedule maintenance events for individual pieces of safety equipment, e.g., negative pressure re-usable respirators 13, to ensure compliance with any procedures or regulations. PPEMS 6 may allow users 20, 24 to create and complete digital checklists with respect to the maintenance procedures and to synchronize any results of the procedures from computing devices 16, 18 to PPEMS 6.

PPEMS 6 provides an integrated suite of personal safety protection equipment management tools and implements various techniques of this disclosure. That is, PPEMS 6 provides an integrated, end-to-end system for managing personal protection equipment, e.g., respirators, used by workers 10 within one or more physical environments 8. The techniques of this disclosure may be realized within various parts of system 2.

PPEMS 6 may integrate an event processing platform configured to process thousand or even millions of concurrent streams of events from digitally enabled devices, such as sensing stations 21, beacons 17, negative pressure re-usable respirators 13, and/or data hubs 14. An underlying analytics engine of PPEMS 6 may apply models to the inbound streams to compute assertions, such as identified anomalies or predicted occurrences of safety events based on conditions or behavior patterns of workers 10.

Further, PPEMS 6 may provide real-time alerting and reporting to notify workers 10 and/or users 20, 24 of any predicted events, anomalies, trends, and the like based on sensed physiological data and/or additional data. The analytics engine of PPEMS 6 may, in some examples, apply analytics to identify relationships or correlations between sensed worker data, environmental conditions, geographic regions and other factors and analyze the impact on safety events. PPEMS 6 may determine, based on the data acquired across populations of workers 10, which particular activities, possibly within certain geographic region, lead to, or are predicted to lead to, unusually high occurrences of safety events.

In this way, PPEMS 6 tightly integrates comprehensive tools for managing personal protective equipment with an underlying analytics engine and communication system to provide data acquisition, monitoring, activity logging, reporting, behavior analytics and alert generation. Moreover, PPEMS 6 provides a communication system for operation and utilization by and between the various elements of system 2. Users 20, 24 may access PPEMS 6 to view results on any analytics performed by PPEMS 6 on data acquired from workers 10. In some examples, PPEMS 6 may present a web-based interface via a web server (e.g., an HTTP server) or client-side applications may be deployed for devices of computing devices 16, 18 used by users 20, 24, such as desktop computers, laptop computers, mobile devices such as smartphones and tablets, or the like.

In some examples, PPEMS 6 may provide a database query engine for directly querying PPEMS 6 to view acquired safety data, compliance data and any results of the analytic engine, e.g., by the way of dashboards, alert notifications, reports and the like. That is, users 20, 24 or software executing on computing devices 16, 18, may submit queries to PPEMS 6 and receive data corresponding to the queries for presentation in the form of one or more reports or dashboards. Such dashboards may provide various insights regarding system 2, such as baseline (“normal”) operation across worker populations, identifications of any anomalous workers engaging in abnormal activities that may potentially expose the worker to risks, identifications of any geographic regions within environments 8 for which unusually anomalous (e.g., high) safety events have been or are predicted to occur, identifications of any of environments 8 exhibiting anomalous occurrences of safety events relative to other environments, and the like.

PPEMS 6 may simplify workflows for individuals charged with monitoring and ensure safety compliance for an entity or environment. That is, PPEMS 6 may enable active safety management and allow an organization to take preventative or correction actions with respect to certain regions within environments 8, particular pieces of safety equipment or individual workers 10, define and may further allow the entity to implement workflow procedures that are data-driven by an underlying analytical engine.

As one example, the underlying analytical engine of PPEMS 6 may be configured to compute and present customer-defined metrics for worker populations within a given environment 8 or across multiple environments for an organization as a whole. For example, PPEMS 6 may be configured to acquire data and provide aggregated performance metrics and predicted behavior analytics across a worker population (e.g., across workers 10 of either or both of environments 8A, 8B). Furthermore, users 20, 24 may set benchmarks for occurrence of any safety incidences, and PPEMS 6 may track actual performance metrics relative to the benchmarks for individuals or defined worker populations.

As another example, PPEMS 6 may further trigger an alert if certain combinations of physiological conditions are present, e.g., to accelerate examination of the worker for safety concerns or rescue, or to service safety equipment, such as one of negative pressure re-usable respirators 13. In this manner, PPEMS 6 may identify individual negative pressure re-usable respirators 13 or workers 10 for which the metrics do not meet the benchmarks and prompt the users to intervene and/or perform procedures to improve the metrics relative to the benchmarks, thereby ensuring compliance and actively managing safety for workers 10.

In some examples, each contaminant capture device 23 includes a communication unit that is configured to transmit information indicative of the respective contaminant capture device 23 to a computing system. For example, the communication device may include an RFID tag configured to output identification information (e.g., a unique identifier, a type of contaminant capture device, etc.) for the respective contaminant capture device 23. In some instances, PPEMS 6 determines whether contaminant capture device 23A is configured to protect worker 10A from hazards within the work environment 8B based on the identification information. For instance, PPEMS 6 may determine the types of contaminants that contaminant capture device 23A is configured to protect against based on a type of the contaminant capture device 23A and compare such types of contaminants to types of contaminants within the work environment 8B. In some examples, the PPEMS 6 alerts worker 10A when the contaminant capture device 23A is not configured to protect workers from contaminants within the work environment 8B, which may enable a worker to utilize the correct contaminant capture device for the hazards within the environment, thereby potentially increasing worker safety.

While described with reference to PPEMS 6, the functionality described in this disclosure may be performed by other computing devices, such as one or more hubs 14 or computing devices of one or more negative pressure re-usable respirators 13. Furthermore, it is also possible to integrate a computing device with the breath sensor, which may be especially useful to in environments where wireless communication is difficult or impossible. In this case, the sensor itself may include processing capabilities and the sensor itself may generate an alert (e.g. and alarm, vibration, or other alert) to the user when the metric indicative of core body temperature indicates a potential problem.

The techniques of this disclosure may enable a computing system to more accurately or timely determine when any of workers 10 are distress, when physiological data indicates problems with the user's air or sudden changes in core body temperature, or when the user may otherwise need assistance. Worker distress may be identified based on one or many sensed conditions described herein. Physiological parameters of the worker, in particular, may be very useful in early detection of worker distress. In addition, physiological parameters of the worker may also be used, in some cases, to identify situations where filter replacement is needed in the respirator device.

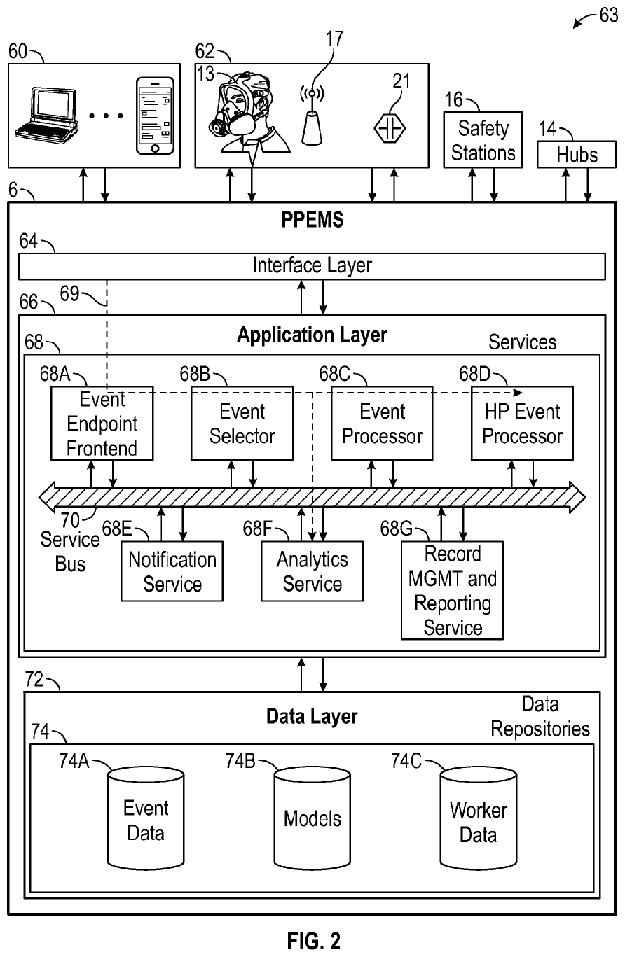

FIG. 2 is a block diagram providing an operating perspective of PPEMS 6 when hosted as a cloud-based system for analytical processing of and/or taking action in response to physiological data sensed from one or more breath sensors embedded within respirator devices used by workers 10 in accordance with techniques described herein. In the example of FIG. 2, the components of PPEMS 6 are arranged according to multiple logical layers that implement the techniques of the disclosure. Each layer may be implemented by one or more modules comprised of hardware, software, or a combination of hardware and software.

In FIG. 2, safety equipment 62 include personal protective equipment (PPE) 13, beacons 17, and sensing stations 21. In some examples, PPE 13 may generally represent a plurality of PPEs in a particular setting or settings. Safety equipment 62, HUBs 14, safety stations 15, as well as computing devices 60, operate as clients 63 that communicate with PPEMS 6 via interface layer 64. Again, although PPE 13 is illustrated as being a negative pressure re-usable respirator, the illustration is only one example and PPE 13 may generally refer to type of PPE respirator device that includes one or more a breath sensors as described herein.

Computing devices 60 typically execute client software applications, such as desktop applications, mobile applications, and web applications. Computing devices 60 may represent any of computing devices 16, 18 of FIG. 1. Examples of computing devices 60 may include, but are not limited to a portable or mobile computing device (e.g., smartphone, wearable computing device, tablet), laptop computers, desktop computers, smart television platforms, and servers, to name only a few examples.

Client applications executing on computing devices 60 may communicate with PPEMS 6 to send and receive data that is retrieved, stored, generated, and/or otherwise processed by services 68. For instance, the client applications may request and edit safety event data including analytical data stored at and/or managed by PPEMS 6. In some examples, client applications may request and display aggregate safety event data that summarizes or otherwise aggregates numerous individual instances of safety events and corresponding data obtained from safety equipment 62 and/or generated by PPEMS 6. The client applications may interact with PPEMS 6 to query for analytics data about past and predicted safety events, behavior trends of workers 10, to name only a few examples. In some examples, the client applications may output for display data received from PPEMS 6 to visualize such data for users of clients 63. As further illustrated and described in below, PPEMS 6 may provide data to the client applications, which the client applications output for display in user interfaces.

Client applications executing on computing devices 60 may be implemented for different platforms but include similar or the same functionality. For instance, a client application may be a desktop application compiled to run on a desktop operating system or a mobile application compiled to run on a mobile operating system. As another example, a client application may be a web application such as a web browser that displays web pages received from PPEMS 6. In the example of a web application, PPEMS 6 may receive requests from the web application (e.g., the web browser), process the requests, and send one or more responses back to the web application. In this way, the collection of web pages, the client-side processing web application, and the server-side processing performed by PPEMS 6 collectively provides the functionality to perform techniques of this disclosure. Accordingly, client applications may use various services of PPEMS 6 in accordance with techniques of this disclosure, and the applications may operate within various different computing environment (e.g., embedded circuitry or processor of a PPE, a desktop operating system, mobile operating system, or web browser, to name only a few examples).

As shown in FIG. 2, PPEMS 6 includes an interface layer 64 that represents a set of application programming interfaces (API) or protocol interface presented and supported by PPEMS 6. Interface layer 64 initially receives messages from any of clients 63 for further processing at PPEMS 6. Interface layer 64 may therefore provide one or more interfaces that are available to client applications executing on clients 63. In some examples, the interfaces may be application programming interfaces (APIs) that are accessible over a network. Interface layer 64 may be implemented with one or more web servers. The one or more web servers may receive incoming requests, process and/or forward data from the requests to services 68, and provide one or more responses, based on data received from services 68, to the client application that initially sent the request. In some examples, the one or more web servers that implement interface layer 64 may include a runtime environment to deploy program logic that provides the one or more interfaces. As further described below, each service may provide a group of one or more interfaces that are accessible via interface layer 64.

In some examples, interface layer 64 may provide Representational State Transfer (RESTful) interfaces that use HTTP methods to interact with services and manipulate resources of PPEMS 6. In such examples, services 68 may generate JavaScript Object Notation (JSON) messages that interface layer 64 sends back to the client application 61 that submitted the initial request. In some examples, interface layer 64 provides web services using Simple Object Access Protocol (SOAP) to process requests from client applications 61. In still other examples, interface layer 64 may use Remote Procedure Calls (RPC) to process requests from clients 63. Upon receiving a request from a client application to use one or more services 68, interface layer 64 sends the data to application layer 66, which includes services 68.

As shown in FIG. 2, PPEMS 6 also includes an application layer 66 that represents a collection of services for implementing much of the underlying operations of PPEMS 6. Application layer 66 receives data included in requests received from client applications 61 and further processes the data according to one or more of services 68 invoked by the requests. Application layer 66 may be implemented as one or more discrete software services executing on one or more application servers, e.g., physical or virtual machines. That is, the application servers provide runtime environments for execution of services 68. In some examples, the functionality interface layer 64 as described above and the functionality of application layer 66 may be implemented at the same server.

Application layer 66 may include one or more separate software services 68, e.g., processes that communicate, e.g., via a logical service bus 70 as one example. Service bus 70 generally represents logical interconnections or set of interfaces that allows different services to send messages to other services, such as by a publish/subscription communication model. For instance, each of services 68 may subscribe to specific types of messages based on criteria set for the respective service. When a service publishes a message of a particular type on service bus 70, other services that subscribe to messages of that type will receive the message. In this way, each of services 68 may communicate data to one another. As another example, services 68 may communicate in point-to-point fashion using sockets or other communication mechanisms. Before describing the functionality of each of services 68, the layers are briefly described herein.

Data layer 72 of PPEMS 6 represents a data repository that provides persistence for data in PPEMS 6 using one or more data repositories 74. A data repository, generally, may be any data structure or software that stores and/or manages data. Examples of data repositories include but are not limited to relational databases, multi-dimensional databases, maps, and hash tables, to name only a few examples. Data layer 72 may be implemented using Relational Database Management System (RDBMS) software to manage data in data repositories 74. The RDBMS software may manage one or more data repositories 74, which may be accessed using Structured Query Language (SQL). Data in the one or more databases may be stored, retrieved, and modified using the RDBMS software. In some examples, data layer 72 may be implemented using an Object Database Management System (ODBMS), Online Analytical Processing (OLAP) database or other suitable data management system.

As shown in FIG. 2, each of services 68A-68G (collectively, services 68) is implemented in a modular form within PPEMS 6. Although shown as separate modules for each service, in some examples the functionality of two or more services may be combined into a single module or component. Each of services 68 may be implemented in software, hardware, or a combination of hardware and software. Moreover, services 68 may be implemented as standalone devices, separate virtual machines or containers, processes, threads or software instructions generally for execution on one or more physical processors. In some examples, one or more of services 68 may each provide one or more interfaces that are exposed through interface layer 64. Accordingly, client applications of computing devices 60 may call one or more interfaces of one or more of services 68 to perform techniques of this disclosure.

In accordance with techniques of the disclosure, services 68 may include an event processing platform including an event endpoint frontend 68A, event selector 68B, event processor 68C, high priority (HP) event processor 68D, notification service 68E, and analytics service 68F.

Event endpoint frontend 68A operates as a frontend interface for exchanging communications with hubs 14, safety stations 15, and safety equipment 62. In other words, event endpoint frontend 68A operates as a frontline interface to safety equipment deployed within environments 8 and utilized by workers 10. In some instances, event endpoint frontend 68A may be implemented as a plurality of tasks or jobs spawned to receive individual inbound communications of event streams 69 that include data sensed and captured by the safety equipment 62. For instance, event streams 69 may include sensor data, such as PPE sensor data from one or more negative pressure re-usable respirators 13, breath temperature data from one or more sensors within re-usable respirators 13, humidity, ambient temperature, and environmental data from one or more sensing stations 21. When receiving event streams 69, for example, event endpoint frontend 68A may spawn tasks to quickly enqueue an inbound communication, referred to as an event, and close the communication session, thereby providing high-speed processing and scalability. Each incoming communication may, for example, carry data recently captured data representing sensed conditions, motions, temperatures, actions or other data, generally referred to as events. Communications exchanged between the event endpoint frontend 68A and safety equipment 62 and/or hubs 14 may be real-time or pseudo real-time depending on communication delays and continuity.

Event selector 68B operates on the stream of events 69 received from safety equipment 62 and/or hubs 14 via frontend 68A and determines, based on rules or classifications, priorities associated with the incoming events. For example, safety rules may indicate that incidents of incorrect equipment for a given environment, incorrect usage of PPEs, rises in core body temperature over a threshold, sudden changes or acceleration in changes of core body temperature, or lack of sensor data associated with a worker's vital signs are to be treated as high priority events. Based on the priorities, event selector 68B enqueues the events for subsequent processing by event processor 68C or high priority (HP) event processor 68D. Additional computational resources and objects may be dedicated to HP event processor 68D so as to ensure responsiveness to critical events, such as incorrect usage of PPEs, lack of vital signs, or other critical events. Responsive to processing high priority events, HP event processor 68D may immediately invoke notification service 68E to generate alerts, instructions, warnings or other similar messages to be output to safety equipment 62, hubs 14, or devices used by users 20, 24. Events not classified as high priority are consumed and processed by event processor 68C.

In general, event processor 68C or high priority (HP) event processor 68D operate on the incoming streams of events to update event data 74A within data repositories 74. In general, event data 74A may include all or a subset of data generated by safety equipment 62. For example, in some instances, event data 74A may include entire streams of data obtained from negative pressure re-usable respirator 13, sensing stations 21, etc. In other instances, event data 74A may include a subset of such data, e.g., associated with a particular time period.

Event processors 68C, 68D may create, read, update, and delete event data stored in event data 74A. Event data for may be stored in a respective database record as a structure that includes name/value pairs of data, such as data tables specified in row/column format. For instance, a name (e.g., column) may be “workerID” and a value may be an employee identification number. An event record may include data such as, but not limited to: worker identification, acquisition timestamp(s) and sensor data. For example, event stream 69 for one or more sensors described herein associated with a given worker (e.g., worker 10A) may be formatted as follows:

{“eventTime”: “2015-12-31T18:20:53.1210933Z”_{;}
“workerID”:“00123”,
“RespiratorType”:“Model 600”,
“ContaminantCaptureDeviceType”: “P90X”,
“ExhalationBreathTemperature”.“35”
“InhalationBreathTemperature”.“27.2”
“HumidityLevel”.“70”,
“HeartRate”.“70”
“Respiratory Rate”:“20”
}

which conveys sensed data of an exhalation breath temperature of 35 degrees Celsius, an inhalation breath temperature of 27.2 degrees Celsius, a relative humidity of 70 percent, a heart rate of 70 beats per minute, and a respiratory rate of 20 breaths per minute, for the captured sensing event. Using this data, for example, a computing device may be able to calculate a metric indicative of core body temperature, which for example, based on the data above may comprise an estimate of core body temperature of 99.1 degrees Fahrenheit.

In some examples, event stream 69 include category identifiers (e.g., “eventTime”, “workerID”, “RespiratorType”, “ContaminantCaptureDeviceType”, and “ExhalationBreathTemperature”, “InhalationBreathTemperature”, “HumidityLevel”, “HeartRate”, and “RespiratoryRate”), as well as corresponding values for each category. Although not shown in the formatted data above, an event stream may also include sensed physiological data or calculated physiological conditions of the worker. These or other formats may be used to communicate a wide range of sensed data.

In some examples, analytics service 68F is configured to perform in depth processing of the incoming stream of events to perform real-time analytics. In this way, stream analytic service 68F may be configured to detect anomalies, transform incoming event data values, trigger alerts upon detecting safety concerns based on conditions or worker behaviors. In addition, stream analytic service 68F may generate output for communicating to safety equipment 62, safety stations 15, hubs 14, or computing devices 60.

Record management and reporting service (RMRS) 68G processes and responds to messages and queries received from computing devices 60 via interface layer 64. For example, record management and reporting service 68G may receive requests from client computing devices for event data related to individual workers, populations or sample sets of workers, geographic regions of environments 8 or environments 8 as a whole, individual or groups (e.g., types) of safety equipment 62. In response, record management and reporting service 68G accesses event information based on the request. Upon retrieving the event data, record management and reporting service 68G constructs an output response to the client application that initially requested the information. In some examples, the data may be included in a document, such as an HTML document, or the data may be encoded in a JSON format or presented by a dashboard application executing on the requesting client computing device. For instance, as further described in this disclosure, example user interfaces that include the event information are depicted in the figures.

As additional examples, record management and reporting service 68G may receive requests to find, analyze, and correlate PPE event information. For instance, record management and reporting service 68G may receive a query request from a client application for event data 74A over a historical time frame, such as a user can view PPE event information over a period of time and/or a computing device can analyze the PPE event information over the period of time.

In accordance with techniques of this disclosure, in some examples, analytics service 68F applies one or more of models 74B to event data 74A representing streams of sensor data from safety equipment 62 to determine whether a worker is in distress, tired, ill, overheating, experiencing hypothermia, or otherwise in need of assistance. In some examples, the sensor data received from safety equipment 62 includes physiological sensor data generated by one or more breath sensors associated with a worker 10, which may be very effective in detecting core body temperature-related health concerns with the worker due to the work environment.

In some examples, the one or more rules are stored in models 74B. Although other technologies can be used, in some examples, the one or more rules are generated using machine learning. In other words, in one example implementation, analytics service 68F utilizes machine learning when operating on event streams 69 so as to perform real-time analytics. That is, analytics service 68F may include executable code generated by application of machine learning. The executable code may take the form of software instructions or rule sets and is generally referred to as a model that can subsequently be applied to event streams 69. The rules, for example, may include rules that are based on sensed breath temperature, sensed physiological data of the user or measured physiological conditions of the user that are calculated based on the sensed physiological data.

Example machine learning techniques that may be employed to generate models 74B can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, the Apriori algorithm, K-Means Clustering, k-Nearest Neighbor (kNN), Learning Vector Quantization (LUQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

Analytics service 68F generates and/or stores, in some example, separate models for individual workers, a population of workers, a particular environment, a type of respirator, a type of contaminant capture device, or combinations thereof. Analytics service 68F may update the models based on sensor data generated by physiological sensors, PPE sensors or environmental sensors. For example, analytics service 68F may update the models for individual workers, a population of workers, a particular environment, a type of respirator, a type of contaminant capture device, or combinations thereof based on data received from safety equipment 62.

In accordance with one or more aspects of this disclosure, in some examples, analytics service 68F applies models 74B to event data 74A to determine whether sensed data from sensors associated with one or more negative pressure re-usable respirators 13 satisfies safety criteria set forth in one or more safety rules associated with a worker. In one example, analytics service 68F determines whether physiological data from sensors within negative pressure re-usable respirator 13A by worker 10A satisfies a safety and/or usage rule based at least in part on worker data 74C, models 74B, event data 74A (e.g., sensor data), or a combination therein.

In some examples, notification service 68E outputs a notification in response to determining that a safety rule is not satisfied (e.g., physiological data of a worker 10 (such as core body temperature) does not satisfy a safety rule, or an article of PPE or component of an article of PPE does not satisfy a safety rule). For example, notification service 68E may output the notification to at least one of clients 63 (e.g., one or more of computing devices 60, hubs 14, safety stations 15, or a combination therein). In some examples, the notification indicates whether sensed physiological parameters of a worker satisfies the one or more rules, e.g., to identify a worker safety event. The notification may indicate which worker of workers 10 is associated with the event (e.g., worker safety concern or the need to replace a filter), a location of the worker, or other worker identification data.

In some examples, notification service 68E outputs a notification in response to determining that physiological parameters of the worker (e.g., core body temperature) indicate a potential problem. For example, notification service 68E may output the notification to at least one of clients 63 (e.g., one or more of computing devices 60, hubs 14, safety stations 15, or a combination therein). In some examples, the notification indicates that physiological parameters of the worker may indicate a potential problem, such as high core body temperature, low core body temperature, sudden changes in core body temperature, or acceleration in the changes to core body temperature. The notification may indicate which worker of workers 10 is associated with the potential problem, a location of the worker, or other identification data.

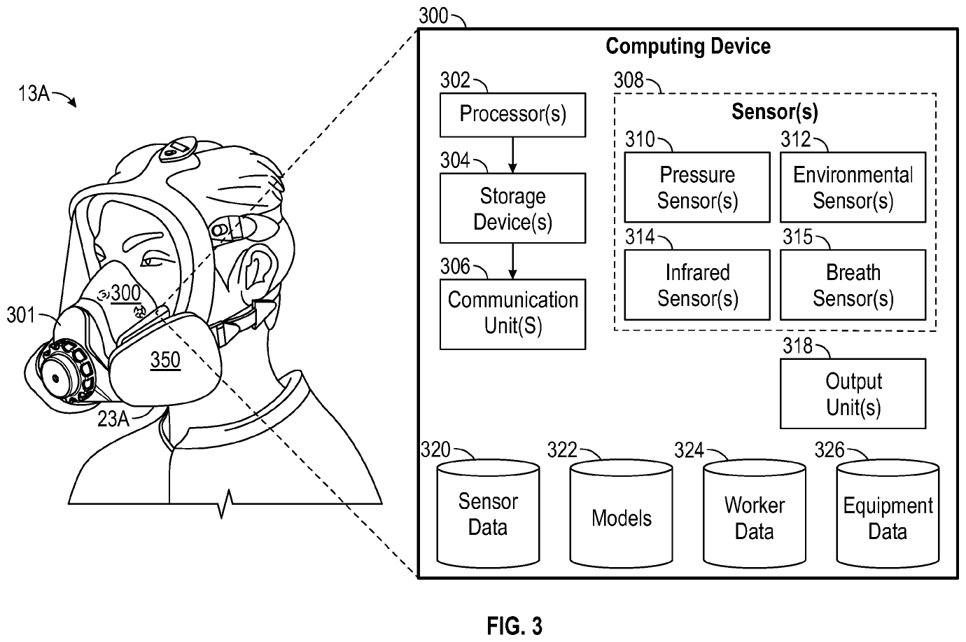

FIG. 3 is a conceptual diagram illustrating an example negative pressure re-usable respirator, in accordance with aspects of this disclosure. Negative pressure re-usable respirator 13A is one example of a PPE respirator device consistent with this disclosure, although this disclosure more generally applies to a wide variety of PPE respirator devices. Negative pressure re-usable respirator 13A is configured to receive (e.g., be physically coupled to) one or more contamination capture devices 23A, such as a particulate filter, a chemical cartridge, or both. Negative pressure re-usable respirator 13A is configured to physically couple to computing device 300. Negative pressure re-usable respirator 13A includes a facepiece (e.g., a full facepiece, or a half facepiece) 301 configured to cover at least a worker's nose and mouth. In some examples, computing device 300 is located with facepiece 301. It should be understood that the architecture and arrangement of negative pressure re-usable respirator 13A and computing device 300 illustrated in FIG. 3 is shown for exemplary purposes only. In other examples, negative pressure re-usable respirator 13A and computing device 300 may be configured in a variety of other ways having additional, fewer, or alternative components than those shown in FIG. 3.

In FIG. 3, negative pressure re-usable respirator 13A may include one or more breath sensor(s) 315 that are positioned to measure inhalation breath temperature and exhalation breath temperature of a user while the user is wearing the PPE respirator device. More specifically, the breath sensor(s) 315 of negative pressure re-usable respirator 13A may be positioned in proximity to inhalation and exhalation valves of a facepiece associated with negative pressure re-usable respirator 13A. Such sensors may be inside the sealable space of a molded body of the facepiece associated with negative pressure re-usable respirator 13A or just inside or just outside of inhalation and exhalation valves associated with the facepiece. In this way, the breath sensor(s) 315 may be integrated with respirator 13A and arranged to promote comfort to the user, while still being effective for sensing inhalation and exhalation breath temperature for use in estimating core body temperature of the user.

FIG. 3 is an example of a PPE respirator device that includes an associated computing device 300, which may provide local processing capabilities for processing sensed data, such as sensed breath temperature data. In the example of FIG. 3, contamination capture device 23A includes a memory device and a communication device, such as RFID tag (e.g., passive RFID tag) 350. RFID tag 350 stores information corresponding to contaminant capture device 23A (e.g., information identifying a type of the contaminant capture device 23A) and outputs the information corresponding to contaminant capture device 23A in response to receiving a signal from another communication device (e.g., an RFID reader).

Computing device 300 may be configured to physically couple to negative pressure re-usable respirator 13A. In some examples, computing device 300 may be disposed between facepiece 301 of negative pressure re-usable respirator 13A and a face of worker 10A. For example, computing device 300 may be physically coupled to an inner wall of the respirator cavity. Computing device 300 may be integral with negative pressure re-usable respirator 13A or physically separable from negative pressure re-usable respirator 13A. In some examples, computing device 300 is physically separate from negative pressure re-usable respirator 13A and communicatively coupled to negative pressure re-usable respirator 13A. For example, computing device 300 may be a smartphone carried by worker 10A or a data hub worn by worker 10A.

Computing device 300 includes one or more processors 302, one or more storage devices 304, one or more communication units 306, one or more sensors 308, one or more output units 318, sensor data 320, models 322, and worker data 324. Processors 302, in one example, are configured to implement functionality and/or process instructions for execution within computing device 300. For example, processors 302 may be capable of processing instructions stored by storage device 304. Processors 302 may include, for example, microprocessors, digital signal processors (DSPs), application specific integrated circuits (ASICs), field-programmable gate array (FPGAs), or equivalent discrete or integrated logic circuitry.

Storage device 304 may include a computer-readable storage medium or computer-readable storage device. In some examples, storage device 304 may include one or more of a short-term memory or a long-term memory. Storage device 304 may include, for example, random access memories (RAM), dynamic random access memories (DRAM), static random access memories (SRAM), magnetic hard discs, optical discs, flash memories, or forms of electrically programmable memories (EPROM) or electrically erasable and programmable memories (EEPROM).

In some examples, storage device 304 may store an operating system or other application that controls the operation of components of computing device 300. For example, the operating system may facilitate the communication of data from electronic sensors 308 to communication unit 306. In some examples, storage device 304 is used to store program instructions for execution by processors 302. Storage device 304 may also be configured to store information within computing device 300 during operation.

Computing device 300 may use one or more communication units 306 to communicate with external devices via one or more wired or wireless connections. Communication units 306 may include various mixers, filters, amplifiers and other components designed for signal modulation, as well as one or more antennas and/or other components designed for transmitting and receiving data. Communication units 306 may send and receive data to other computing devices using any one or more suitable data communication techniques. Examples of such communication techniques may include TCP/IP, Ethernet, Wi-Fi, Bluetooth, 4G, LTE, to name only a few examples. In some instances, communication units 306 may operate in accordance with the Bluetooth Low Energy (BLU) protocol. In some examples, communication units 306 may include a short-range communication unit, such as an RFID reader.

In general, computing device 300 includes a plurality of sensors 308 that generate sensor data indicative of operational characteristics of negative pressure re-usable respirator 13A, contaminant capture devices 23A, and/or an environment in which negative pressure re-usable respirator 13A is used. Sensors 308 may include an accelerometer, a magnetometer, an altimeter, an environmental sensor, among other examples. In some examples, environment sensors may include one or more sensors configured to measure temperature, humidity, particulate content, gas or vapor concentration levels, or any variety of other characteristics of environments in which negative pressure re-usable respirator 13A are used. In some examples, one or more of sensors 308 may be disposed between facepiece 301 of negative pressure re-usable respirator 13A and a face of worker 10A. For example, one of sensors 308 (e.g., an air pressure sensor) may be physically coupled to an inner wall of the respirator cavity.

In the example of FIG. 3, sensors 308 include one or more air pressure sensors 310 configured to measure air pressure within a cavity formed or defined by a face of worker 10A and negative pressure re-usable respirator 13A. In other words, air pressure sensors 310 detect the air pressure of the air located in the sealable space between the face of worker 10A and facepiece 301 as the worker inhales and exhales.

In addition, sensors 308 include one or more breath sensor(s) 315 positioned to measure exhalation breath temperature and inhalation breath temperature of a user while the user is wearing negative pressure re-usable respirator 13A. More specifically, the breath sensor(s) 315 of negative pressure re-usable respirator 13A may be positioned inside (or outside) the sealable space of a molded body of the facepiece associated with negative pressure re-usable respirator 13A in proximity to inhalation and exhalation valves, and the breath sensor(s) 315 may be configured to sense inhalation and exhalation breath temperature of a user. In this way, the breath sensor(s) 315 is arranged to promote comfort to the user, while still being effective for sensing breath temperature, which may be used to generate a metric (such as an estimate) of the user's core body temperature.

Computing device 300 includes one or more output units 318 configured to output data that is indicative of operation of negative pressure re-usable respirator 13A. In some examples, output unit 318 output data from the one or more sensors 308 of negative pressure re-usable respirator 13A. For example, output unit 318 may generate one or more messages containing real-time or near real-time data from one or more sensors 308 of negative pressure re-usable respirator 13A for transmission to another device via communication unit 306. In some examples, output unit 318 are configured to transmit the sensor data in real-time or near-real time to another processing device (e.g., a processing device of HUB 14 or PPEMS 6) via communication unit 306. However, in some instances, communication unit 306 may not be able to communicate with such devices, e.g., due to an environment in which negative pressure re-usable respirator 13A is located and/or network outages. In such instances, output unit 318 may cache usage data to storage device 304. That is, output unit 318 (or the sensors themselves) may send usage data to storage device 304, e.g., as sensor data 320, which may allow the usage data to be uploaded to another device upon a network connection becoming available.

In some examples, output unit 318 is configured to generate an audible, visual, tactile, or other output that is perceptible by a user of negative pressure re-usable respirator 13A. Examples of output are audio, visual, or tactile output. For example, output units 318 include one more user interface devices including, as examples, a variety of lights, displays, haptic feedback generators, speakers or the like. Output units 318 may interpret received alert data and generate an output (e.g., an audible, visual, or tactile output) to notify a worker using negative pressure re-usable respirator 13A of an alert condition (e.g., that the likelihood of a safety event is relatively high, that the environment is dangerous, that negative pressure re-usable respirator 13A is malfunctioning, that one or more components of negative pressure re-usable respirator 13A need to be repaired or replaced, or to alert for another reason).

According to aspects of this disclosure, processors 302 utilize sensor data (e.g., data from pressure sensors 310, environmental sensors 312, and/or infrared sensors 314 of computing device 300, data from sensing stations 21 of FIG. 1, or other sensors such as breath sensor(s) 315) in a variety of ways. In some examples, processors 302 are configured to perform all or a portion of the functionality of PPEMS 6 described in FIGS. 1 and 2. While processors 302 are described as performing the functionality in FIG. 3, in some examples, other devices (e.g., PPEMS 6, hubs 14, other devices, or a combination therein) perform functionality described with reference to processors 302.

In the example of FIG. 3, computing device 300 includes sensor data 320, models 322, and worker data 324. Sensor data 320 includes data regarding operation of negative pressure re-usable respirator 13A, physiological conditions of worker 10A, characteristics of environment 8B, or a combination thereof. In other words, sensor data 320 may include data from PPE sensors, physiological sensors, breath sensors, and/or environmental sensors. Models 322 include historical data (e.g., historical sensor data) and models, such as models 74B described with reference to FIG. 2. Worker data 324 may include worker profiles, such as worker data 74C described with reference to FIG. 2.

In general, processors 302 applies models 322 to data captured by sensors 310 to detect any anomaly or unsafe condition as to the health and safety of worker 10A based on physiological data and/or other captured data. In some examples, models 322 may be trained based on historical data (e.g., historical physiological sensor data of a given user). For example, models 322 may be trained on historical breath sensor data associated with worker 10A during normal working conditions, which may establish a baseline for that worker. Feedback from worker 10A indicating worker 10A is having difficulty breathing, or feedback from breath sensors indicating changes or deviations from the established baseline for that worker may indicate problems.

In some examples, this disclosure describes a system and method for monitoring and tracking a worker physiology (e.g., a fire fighter, soldier, or industrial worker) while the user is wearing respiratory protection. In some example, the system may comprise an internet of things (IoT) sensor attached to an exhalation port of a negative pressure full face respirator. In some examples, sensors may collect temperature signals at multiple locations within the device including inhaled air temperature and exhaled air temperature. A set of algorithms, deployed on the IoT device or performed by an internal or external computing device, may be used to convert these signals into an estimate of a worker's core body temperature or another metric indicative of core body temperature. The estimated values may be compared to baseline values and/or workplace guidelines, and an actionable alert can be triggered if such values exceed a predetermined threshold.

Exertion heat stress is one of the top environmental risk to soldiers, fire fighters and workers who operate in outdoors environments. Temperature increase due to climate change and military training and operations that are typically conducted in hot environment, which make heat-related injuries a major medical conern for the military. The human body's thermoregulaiton system can typically adjust to meet physiological stresses that occur with exertion. However, this process is impeded when a person is involved in heavy exertion. Exertion heat stress usually occurs when a healthy individual performs physiocal activity in hot environments. Firefighters and soldiers are often required to carry heavy equipment while wearing thermal and flame resistant protective clothing and equipment. The heavy clothing prevent evaporative loss of metabolic heat, while the heavy equipment add to the stress and compound exertion effects. Under such stressful conditions, the soldiers and firefighters may experience a decrease in cardiac output leading to rapid rise in core body temperature that remains even after the termination of the stressful activity. Moreover, core body temperature oftent continues to rise post incident. Therefore, monitoring for signs of heat stress is an important function of safety officers in the workplace and the field. Remote, non-invasive monitoring can play a key role in detecting and preventing adverse health conditions such as heat stress and cardiovascular over exertion.

United States military data, for example, has shown that heat stroke is somewhat common for solders. In addition, government reports have identified hundreds of deaths within less than a decade of time, and thousands of heat related injuries occurring in the United States in some years. The majority of firefighter injuries in are commonly due to exertion heat stress.

Currently, heat stress monitoring is carried out using heat index charts. In rare situations, especially during training for military and firefighters/first responders, wearable heat stress monitoring devices are used. Most conventional methods for tracking core body temperature for heat stress monitoring are highly invasive, requiring either rectal thermometry, ingestible pills, or esophageal probes to track. This problem is further exacerbated by the general variation in measured core temperature depending on the location of the body being monitored. In an ambient condition of 25 C, the exhaled breath of a healthy person is around 35 C while the core body temperature is around 37 C. Some studies have shown that core body temperature of firefighters can rise to 102 F after only 15 minutes of strenuous exercise, and it continues to rise after stopping the exercise peaking about 5 minutes into the resting period. This points to the need for accurate and rea-time monitoring of core body temperature of persons involved in stressful activities such as soldiers and firefighters.

Several products have entered the commercial market in recent years to track core body temperature non-invasively of ambulatory individuals. These fall into two primary categories: in-ear monitors and chest-based harnesses. In-ear monitors attempt to measure information in the ear-canal through either IR thermometry or an embedded thermistor. Chest based systems track a wearer's heart rate and use an algorithm developed by the US Army Research Institute for Environmental Medicine ECTemp. Harness-based physiological monitors are very expensive, costing on the order of thousands of dollars per unit, in addition to software subscription fees. Moreover, these systems are not conforming to current PPE offerings, with earbuds potentially impeding the use of hearing or respiratory protection, while chest harnesses require someone to wear these systems under their normal clothing to gather the appropriate signals (with associated challenges in hygiene, comfort, and fit).

Therefore, there is a need for respirators equipped with a physiological sensor that does not contact the skin of the wearer, but provides accurate measure of the state of health of the wearer, and communicates the data wirelessly to an external hub where the data are analyzed and alarms are sent to the wearer and his/her supervisor if maximum values for core body temperature are exceeded.

Exhaled breath temperature tracks with core body temperature. As air is inhaled, gas exchange in the lungs (via the alveoli) will induce a chance in the temperature of the inspired air towards the temperature of the lungs (effectively core). Due to the surface area/general design of the lungs, rapid thermal exchange can occur allowing for a measurable response to be observed in the expired breath. Laboratory type setups may include complex face masks with tubing and highly sensitive thermocouples to isolate the expired air temperature, or external apparatus that a person would breath into to track their breathing. These options may be well suited for patient monitoring, however, they are not viable for an ambulatory worker. This disclosure provides a simpler system to measure exhaled breath temperature via sensors that are attached to respirators worn by workers.

In some examples, the techniques of this disclosure may apply to reusable respirators in a connected safety environment. In addition, the techniques could also be employed in many other settings, such as hospital settings to augment other patient monitoring systems with a secondary method of tracking a patient's core body temperature that does not require additional electrodes mounted on a patient, if the patient is wearing an air mask or other ventilator device.

At ambient condition of 25 C, the exhaled breath of a healthy person is around 35 C while the core body temperature is around 37 C. Moreover, exhaled breath temperature tracks core body temperature with a fixed offset. The techniques of this disclosure take advantage of this knowledge to design and build a working respirator that measures the exhaled breath temperature of the wearer.

In some examples, this disclosure describes a system and method for monitoring and tracking a worker's physiology while wearing respiratory protection. The system may include an IoT sensor embedded in the nose cup of a respirator capable of collecting raw signals of the pressure, temperature, and humidity of the wearer inside the mask. A set of algorithms, deployed on the IoT device or on a connected external compute device like a phone or hub, may convert these signals into an estimate of a worker's respiration rate, volume of air flowing through the mass, and worker's core body temperature. These estimates can be used in conjunction with a set of workplace guidelines or control limits to trigger intervention when a worker nears unsafe physiological levels.

Wearers of PPE respirator devices operate in particularly hazardous and stressful environments, leading to an industry desire for better monitoring of worker physiology to provide alerts/indications of a worker's wellbeing. An important metric of a worker's health is their core body temperature, which is nominally in the 36 C to 37.5 C range for a healthy individual. If a person undergoes prolonged exposure in a hot environment and/or performs continued strenuous activity, they core body temperature will rise. Tracking this rise is important, as a person who reaches above 39.5 C is at a high likelihood for heat stress and death due to their exposure. Core body temperature values for a person working at 165 W/m2 at 35 C outdoors in sunny weather wearing SCBA and fire fighter suit can be significantly higher than that of the same worker in the same environment in normal clothing.

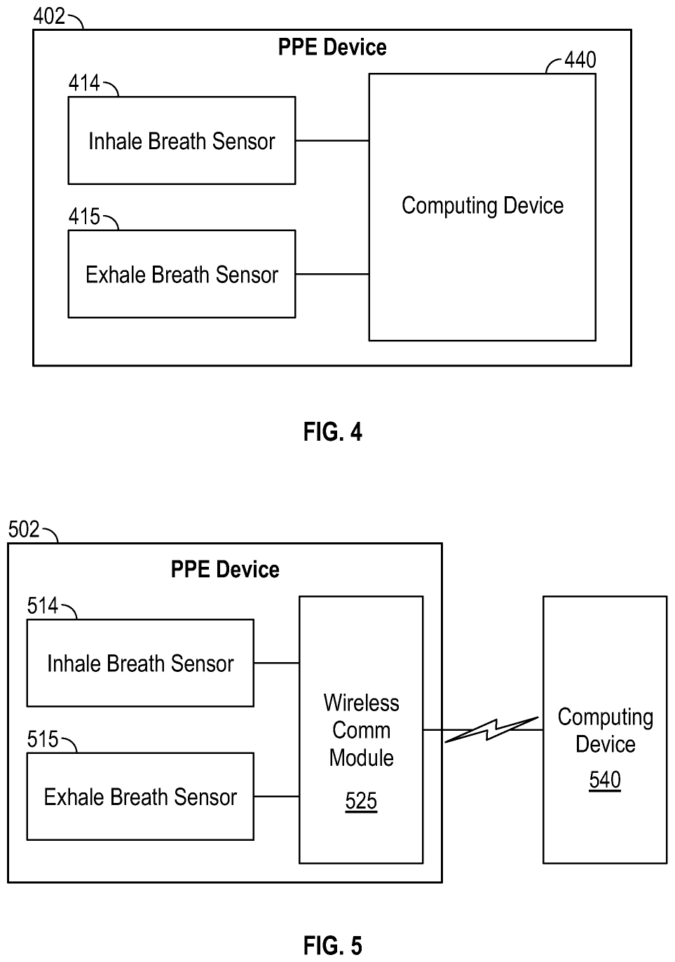

FIGS. 4 and 5 are block diagrams illustrating two example systems showing a PPE respirator device with breath sensors in communication with a computing device. In particular, FIG. 4 shows an example where PPE respirator device 402 includes an inhale breath sensor 414, an exhale breath sensor 415, and a local computing device 440 configured to process sensor data from inhale breath sensor 414 and exhale breath sensor 415. FIG. 5 shows an alternative example where PPE respirator device 502 includes an inhale breath sensor 514 and exhale breath sensor 515 electrically coupled to a wireless communication module 525 to communicate with an external computing device 540 configured to process sensor data from inhale breath sensor 514 and exhale breath sensor 515. In still other cases, inhale breath sensor 514 and exhale breath sensor 515 may each include a wireless communication module (like module 925) as part of an integrated design.

Moreover, in yet additional examples (not shown in FIGS. 4 and 5), the sensors themselves may include computing devices in an integrated design, which may be especially useful in situations or environments where wireless communication is impossible or undesirable. In this case, the computing devices integrated with the sensors may process sensor data, as described herein, to generate a metric indicative of core body temperature, may store core body temperature information for later download, and may issue local alerts (such as alarms or vibration) to the user when problems are detected with core body temperature.

Referring again to the examples of FIGS. 4 and 5, computing device 440 may refer to any computing device that is integrated with a PPE respirator device 402, whereas computing device 540 may refer to any external computing device that is separate from PPE respirator device 502. FIGS. 1-3 describe many such computing devices, and other computing devices may also be used. Again, in other examples, the computing devices for processing sensor data and the sensors may be fully integrated into a self-contained module, such as an IoT sensor device, which may have wireless communication capabilities for sending data after processing the data, or for offloading data at a later time, after storing and processing in an environment that does not allow for wireless communication.

PPE respirator devices 402 and 502 are two examples of respirator devices described herein, which include an exhalation breath sensor 415, 515 arranged to detect exhalation breath temperature of a user. With each of the systems of FIG. 4 or FIG. 5, at least one computing device 440 or 540 is configured to generate a metric indicative of core body temperature of the user based on the detected exhalation breath temperature. In addition to exhalation breath sensor 415, 515, in some examples, PPE respirator devices 402 and 502 may also include an inhalation breath sensor 414, 514 arranged to detect inhalation breath temperature, in which case computing device 440, 540 may generate the metric based on the detected exhalation breath temperature and the detected inhalation breath temperature. In some cases, inhalation breath sensor 414, 515 and/or exhalation breath sensor (or additional sensors) may be further configured to sense other parameters. The other parameters may comprise environmental factors, such as air humidity, or additional physiological parameters of the user, such as respiratory rate. In such cases, computing device 440, 540 may be configured to generate the metric based on the detected exhalation breath temperature, the detected inhalation breath temperature, and the detected humidity. In addition, in such cases, computing device 440, 540 may be configured to generate one or more additional metrics, such as heart rate or breathing rate based on sensed parameters.

The metric may be defined or refined in wide variety of ways. In some examples, the metric comprises an estimation of core body temperature that is based on the measured exhalation breath temperature. In some examples, the metric comprises an estimation of core body temperature that is based on the measured exhalation breath temperature and one or more adjustments. The adjustments, for example, may comprise an offset, which may be fixed or refined based on one or more other factors. Accordingly, in some examples, the metric may comprise the measured exhalation breath temperature with a fixed offset. In still other examples, the adjustments may be based on such things as measured humidity, other ambient measurements, or measured physiological parameters such as heart rate or respiratory rate, or any other factor or combination of factors that could help improve a core body temperature estimate that is based on exhalation breath temperature.

In various examples, in addition to exhalation breath temperature, a number of additional measurements may be used to help define or refine the metric. Such additional measurements may be used to define or refine the metric and may, for example, provide refinements or adjustment to the offset, as mentioned above. Additional measurements, for example, that may be used to define or refine the metric may include internal and differential air pressure measurement to extract breathing dynamics (e.g. breathing rate, inspiration flow rate, expiration flow rate, tidal air volume, residence time of air in the lung, or other breathing dynamics). In order to determine internal and differential air pressure measurements, sensors may be used on the exterior of a PPE respirator device and on the interior (e.g., inside the sealable space of the PPE respirator or inside another insulated location outside of the sealable space yet inside of the PPE respirator, such as just outside of an exhalation valve associated with the PPE device in an insulated region of the PPE respirator device).

Other additional factors or measurements that may be used to define or refine the metric may include subject information such as body mass of the user (e.g., body mass index), sex of the user, baseline user measurements of heart rate, breath rate, or other factors associated with a user. In addition, still other factors that may be used to define or refine the metric may include mask information (i.e., information about the PPE respirator device), such as internal dead space, insulation in interior measurement locations, flow resistance (e.g., due to valves or other components), internal flow patterns, or other mask information. Still other factors that may be used to define or refine the metric indicative of core body temperature may include environmental conditions (e.g., temperature, humidity, air pressure, or other environmental conditions). Also, to help ensure an accurate metric, it may desirable to integrate a known or reference thermal mass of air flow within the flow circuit (e.g., within or around air flow within the PPE respirator device). Moreover, to help ensure an accurate metric, it may desirable to use an insulated and isolated measurement space to provide a more accurate input and output airflow environment, e.g., such as by creating an insulated space within the PPE device just outside of the exhalation port of the sealable space defined in the PPE device.

Factors associated with a user of respirator device (which may be used to adjust or define the metric indicative of core body temperature) may include residence time of air in the user's lungs, height, weight, sex, body mass index, or other factors. Factors associated with the respirator device (which may be used to adjust or define the metric indicative of core body temperature) may include a pressure/flow profile and a temperature profile of inhaled air, a pressure/flow profile and a temperature flow profile of exhaled air, and possibly a humidity level inside the device. Environmental factors (which may be used to adjust or define the metric indicative of core body temperature) may include temperature, humidity, air pressure, levels of air contaminants, or other environmental factors.

In some examples, thermal modeling may be based on things such as mass of air heated, specific heat, air chamber model, models of lungs, trachea, mouth, lung exchange surface, dead space in the trachea, humidification, or other factors. Modeling of physical properties of a respirator device may include things such as insulation, flow patterns, recirculation, dead space, material properties, or other factors.

In addition to generating the metric indicative of core body temperature, computing device 440, 540 may be further configured to generate an alert in response to the metric indicating a potential problem related to core body temperature. For example, computing device 440, 540 may be configured to generate an alert based on the metric indicating core body temperature being over a threshold. In another example, computing device 440, 540 may be configured to generate an alert based on the metric indicating core body temperature being below a threshold.

In some examples, the metric generated by computing device 440, 540 may be based on an absolute difference between the detected inhalation breath temperature and the detected exhalation breath temperature. Computing device 440, 540 may be configured to generate an alert in response to the metric indicating a potential problem related to core body temperature. Moreover, in some example, computing device 440, 540 may be configured to define a baseline of the user based on the detected inhalation breath temperature and the detected exhalation breath temperature during a first time period and to generate the alert in response to the metric changing relative to the baseline in a second time period. The computing device 440, 540, for example may associate the baseline with the user wearing the PPE respirator device, and thus, different users may have different baselines. Changes in the metric relative to the baseline, for example, may include a first derivative of the metric, i.e., a time rate of change of the metric, or possibly a second derivative of the metric, i.e., a rate of acceleration of change to the metric. The derivative and second derivatives of the metric may be helpful to identify situations where core body temperature is changing rapidly, possibly indicating problems or a need to rescue the worker using PPE device 402, 502.

As noted, computing device 440 may be part of PPE respirator device 402, and in other cases the computing device and the sensors may be fully integrated into a computer module. With FIG. 5, however, computing device 540 is external to PPE respirator device 502. In this case, computing device 540 may be associated with the user of the PPE respirator device or may be associated with another user within a connected work environment, such as a supervisor of the user of PPE respirator device 502.

PPE respirator device 402, 502 may comprise any of a wide range of respirator devices. Examples include a full-face respirator device, a half-face respirator device, a negative pressure reusable respirator system, a powered air purifying respirator (PAPR), a filtered face piece respirator (FFR), a respirator that forms part of a self-contained breathing apparatus (SCBA), a respirator that forms part of a safety helmet or mask, a respirator that forms part of firefighter safety equipment, a respirator that forms part of military equipment, and/or a medical respirator device, to name a few.

In many cases, PPE respirator device comprises a facepiece designed to fit over a nose and a mouth of a user, the facepiece defining a sealable space formed around the nose and the mouth of the user for delivering air to the user. For example, the sealable space may be formed of a molded body that presses against the face of a user to define a sealable space around the nose and mouth. Inhalation and exhalation valves may be included in the facepiece to allow air to enter and exit the sealable space. In particular, inhaled air may enter through a contaminant filter and then through an inhalation valve. Exhaled air may exit though an exhalation valve that is different than the inhalation valve.

According to this disclosure, the exhalation breath sensor 415, 515 may be positioned proximate to the exhalation valve of a facepiece of PPE respirator device 402, 502. For example, the exhalation breath sensor 415, 515 may be positioned inside the sealable space defined by a facepiece and proximate to the exhalation valve of the facepiece of the PPE respirator device. More specifically, in some examples, the exhalation breath sensor 415, 515 may be positioned between the nose and/or mouth of the user and the exhalation valve of the PPE respirator device 402, 502. In different examples, the exhalation breath sensor 415, 515 may be positioned in proximity to the exhalation valve of a facepiece either within the sealable space (i.e., on an interior side of the valve) or outside of the sealable space (i.e., on an exterior side of the valve). If on the exterior, the exhalation sensor may be positioned within a vent cap that covers the exterior side of the exhalation valve. The vent cap may also be useful location for housing a wireless communication module 525 that wirelessly communicates sensor data to computing device 540. In some cases, multiple sensors may be used for exhalation breath sensing, which may provide redundancy and possible improvements when one or more sensors are ineffective. In this case, the computing device may parse sensed data from multiple exhalation breath sensors, determine the metric based on multiple versions of the sensed data, and possibly disregard sensed data that is determined to be incorrect or corrupted.

Inhalation sensor 414, 514 may be positioned proximate to an inhalation valve of a facepiece of PPE respirator device 402, 502. For example, the inhalation sensor 414, 514 may be positioned inside the sealable space defined by a facepiece and proximate to the inhalation valve of the facepiece of the PPE respirator device. Alternatively, inhalation sensor 414, 514 sensors may be positioned in proximity to the inhalation valve of a facepiece either within the sealable space (i.e., on an interior side of the valve) or outside of the sealable space (i.e., on an exterior side of the valve). If on the exterior, the exhalation sensor may be positioned on or outside of a contaminant filter, directly on an inlet valve, or possible other locations. In some cases, the inhalation breath sensor may be positioned outside of a sealable space of the facepiece of PPE respirator device 402, 502, while exhalation breath sensor is positioned inside of the sealable space of the facepiece of PPE respirator device 402, 502. In still other examples, an ambient temperature sensor may be used instead of (or in addition to) an inhalation breath sensor, in which case the metric may be generated by a computing device based on detected exhalation breath temperature and ambient temperature (with or without inhalation breath temperature).

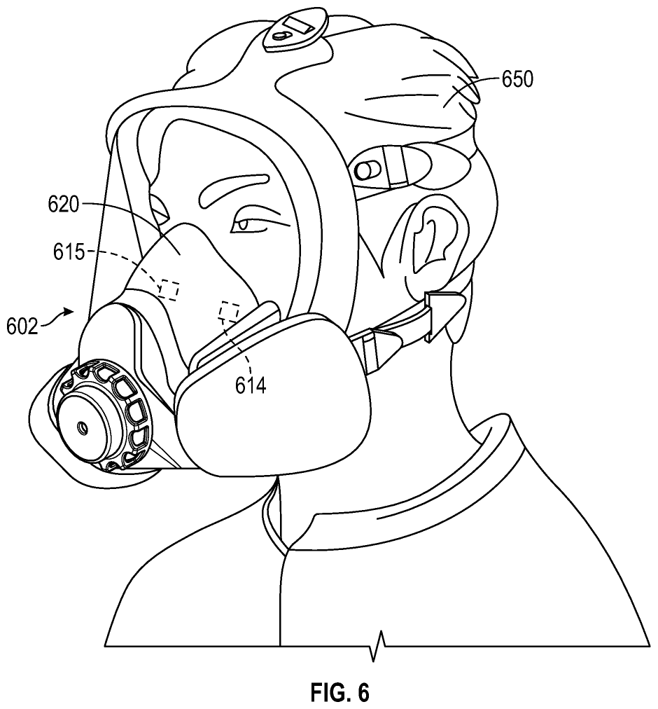

FIG. 6 is a perspective view of an exemplary PPE respirator device 602 worn by a user 650. PPE respirator device 602 is shown as a full-face negative pressure reusable respirator system, although the techniques and devices of this disclosure can apply to a wide variety of respirator devices and systems. As shown, PPE respirator device 602 includes a facepiece 620 comprising a molded body that designed to fit over a nose and a mouth of user 650 so as to define a sealable space formed around the nose and the mouth of user 650 for delivering air to user 650. An exhalation breath sensor 615 is positioned inside the sealable space of facepiece 620. In other words, in this example, exhalation breath sensor 615 is positioned within the sealable space defined by a molded body portion of facepiece 620 and the face of user 650. This interior region of sealable space may comprise a good location for detecting exhaled breath temperature, although many other locations are also possible within PPE respirator device 602.

In some examples, a PPE respirator device 602 comprises a facepiece 620 designed to fit over a nose and a mouth of a user, the facepiece defining a sealable space formed around the nose and the mouth of the user for delivering air to the user, wherein the facepiece includes an inhalation valve (e.g., inside the sealable space and not viewable in FIG. 6) and an exhalation valve (e.g., also inside the sealable space and not viewable in FIG. 6). A first sensor 615 is positioned proximate to the exhalation valve and configured to detect exhalation breath temperature, and a second sensor 615 is positioned proximate to the inhalation valve and configured to detect inhalation breath temperature.

In some cases, PPE respirator device further includes a wireless communication module (not shown in FIG. 6), which may be connected to first sensor 614 and second sensor 615 and wherein the wireless communication module wireless communicates sensor data to a computing device, which may be external to PPE respirator device 602.

The inhalation and exhalation valves or ports of a ventilator facepiece can vary for different ventilator devices. Therefore, the desirable location for placement of breath sensors 614 and 615 may be different for different ventilator devices and different styles or designs of ventilators. In various examples, it may be desirable to have inhalation breath sensors (such as sensor 614) placed near inhalation valves, such as inside the sealable space of a facepiece 620 near the inhalation valve of the facepiece of a ventilator device, or outside the sealable space of the facepiece 620 near the inhalation valve of the facepiece of the ventilator device.

Likewise, it may be desirable to have exhalation breath sensors (such as senor 615) placed near exhalation valves, such as inside the sealable space of the facepiece 620 near the exhalation valve of the facepiece, or outside the sealable space of facepiece 620 near the exhalation valve of the facepiece. The exhalation breath sensor (e.g., sensor 615), in particular, may be especially useful inside the sealable space of the facepiece 620 where air exhaled air is sealed from the environment until it exits the exhalation valve. For example, it may be desirable for the exhalation breath sensor to be positioned inside the sealable space between the exhalation valve of facepiece 620 and the mouth or nose of user 650. This location may be particularly good for accurate exhalation breath temperature measurements.

In some examples, the first sensor (e.g., the exhalation sensor 615) is positioned inside the sealable space proximate to the exhalation valve of the facepiece and wherein the second sensor (i.e., the inhalation sensor 614) is positioned inside the sealable space proximate to the inhalation valve of the facepiece. In another example, the first sensor (e.g., the exhalation sensor) may be positioned outside the sealable space proximate to the exhalation valve of the facepiece and the second sensor (e.g., the inhalation sensor) may be positioned outside the sealable space proximate to the inhalation valve of the facepiece. In still other cases, the first sensor (e.g., the exhalation sensor) may be positioned inside the sealable space proximate to the exhalation valve of the facepiece and the second sensor (e.g., the inhalation sensor) is positioned outside the sealable space proximate to the inhalation valve of the facepiece. Many other configurations and sensor locations are possible. Furthermore, in some cases, the inhalation sensor may be replaced with an ambient sensor located anywhere, in, on or around PPE respirator device 620 insofar as inhaled air should be generally similar to ambient air. Depending on the conditions, however, inhaled air may differ from ambient air insofar as inhaled air may be filtered by PPE device 602 and ambient air may include contaminants. For this reason, a properly placed inhalation sensor may produce better inhalation breath data than an ambient sensor, which can then be processed by a computing device (with the exhalation breath sensor data) to define the core body temperature metric as described herein.

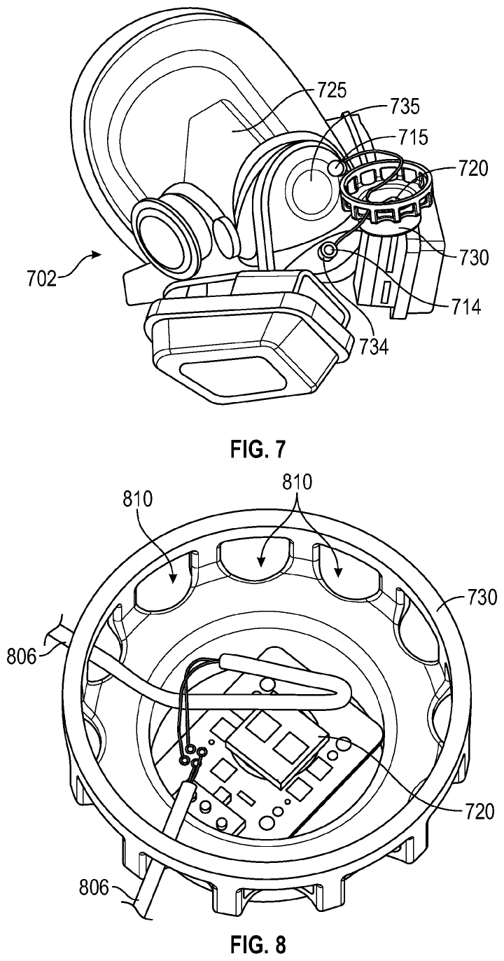

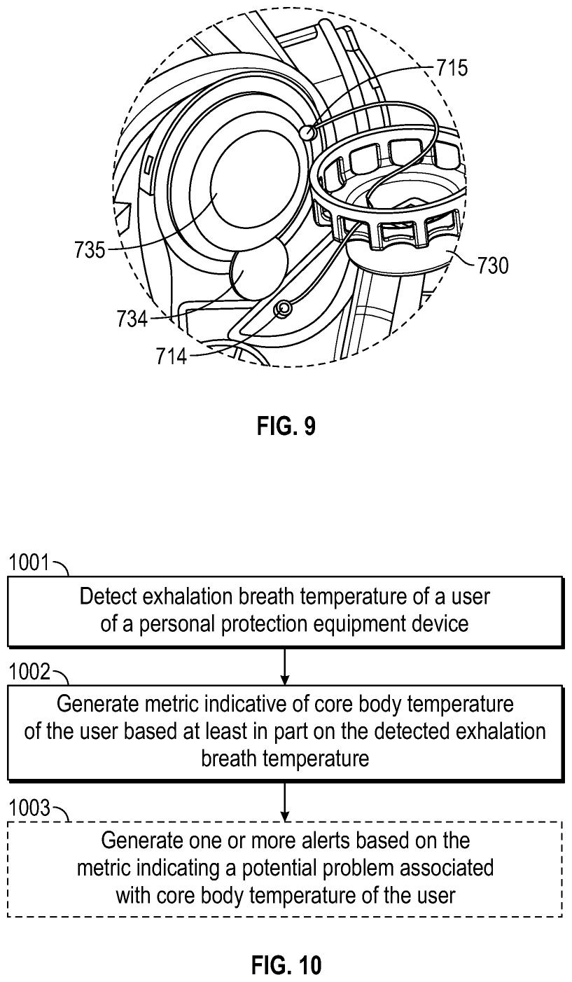

FIG. 7 is a perspective view of an example prototype of a negative pressure re-usable respirator device 702 that includes breath sensors 715 and 714. FIG. 8 is a perspective view inside of a molded cap 730 of prototype of FIG. 7, whereby the molded cap 730 houses a circuit 720 that may form part of a respirator device. FIG. 9 is a perspective disassembled view of the prototype of FIG. 7 showing breath sensors located in proximity to inhalation and exhalation valves (e.g., outside of the sealable space of the facepiece 725) of the negative pressure re-usable respirator device. Although sensor placement may not be ideal in FIGS. 7-9, it was effective to demonstrate proof of concept and a workable prototype.

Example respirator device 702 comprises a facepiece 725, which is designed to fit around a users face and create a sealable space for delivery of air. In particular, facepiece 725 may comprise a molded body designed to fit snugly around a users face, and inhalation valve 734 and exhalation valve 735 allow for air to pass into and exit from the sealable space. For example, air may pass through inhalation valve 734 and through one or more filters before entering the sealable space of facepiece 725. Exhaled air from a user's nose or mouth exits the sealable space (and PPE respirator device 702) via exhalation valve 735. A vent cap 730 (shown disassembled from PPE respirator device 702) may cover exhalation valve 735.

According to this disclosure, PPE device 702 comprises an exhalation sensor 715 positioned to detect exhalation breath temperature. In the prototype design of FIG. 7 exhalation sensor 715 is located near exhalation valve 735 outside of the sealed space of PPE device 702, although this disclosure contemplates many other useful locations for detecting exhalation breath temperature.

PPE device 702 of FIG. 7 also includes an inhalation sensor 714, which for the prototype design, is positioned near inhalation valve 735 outside of the sealed space of PPE device 702, although this disclosure also contemplates other locations for detecting inhalation breath temperature. In some cases, an ambient sensor configured to detect the temperature of ambient air may be used instead of (or in addition to) inhalation sensor 714.

PPE device 702 also includes a circuit 720, electorally coupled to sensors 714 and 715. In the prototype design, circuit 720 is housed inside of vent cap 730, which covers exhalation valve 735. Wires 806 connect circuit 720 to sensors 714 and 715, and wires 806 may pass thought holes 810 of vent cap 730. This type of housing and arrangement of circuit 720 ensures a compact and ergonomic design that does not interfere with airflow or user comfort. In the prototype, circuit 720 comprises a wireless module for receiving and communicating senor data from PPE respirator device 702 to an external computing device for processing and generating the metric indicative of core body temperature. In other examples, circuit 720 could comprise the computing device that processes the sensor data.

For the prototype design, thermistors were used for sensors 714 and 715, i.e., the Murata NXFT15XH103FA2B025 thermistor with specifications set forth in the Table below:

Murata NXFT15XH103FA2B025

| | | | | | Maximum | Rated | Typical | |
|---|---|---|---|---|---|---|---|---|
| | B- | B-Constant | B-Constant | B-Constant | Operating | Electric | Dissipation | Thermal |
| Resistance | Constant | (25-80° C.) | (25-85° C.) | (25-100° C.) | Current | Power | Constant | Time |
| (25° C.) | (25-50° C.) | (Reference | (Reference | (Reference | (25° C.) | (25° C.) | (25° C.) | Constant |
| (ohm) | (K) | Value) (K) | Value) (K) | Value) (K) | (mA) | (mW) | (mW/° C.) | (25° C.) (s) |
| 10k ± | 3380 ± 1% | 3428 | 3434 | 3455 | 0.12 | 7.5 | 1.5 | 4 |
| 1% | | | | | | | | |

For the prototype design, circuit 720 was realized with a Laird BL652-SA-01 module, based on a Nordic nRF52832 Bluetooth chip. These components were mounted on custom board powered via coin cell battery to define circuit 720.

Data acquisition and display using a prototype was performed. In particular, inhale and exhale temperature data acquired from the Bluetooth module was wirelessly transmitted to a platform Bluetooth Gateway and data was displayed for analysis on time-series data visualization system. This prototype enabled further experimentation and understanding of sensor performance as a proxy for heat stress and provide real time notifications. Real time wireless telemetry was also displayed using various augmented reality devices (tablets and wearable headsets). This allowed relevant data to be attached to a user's field of view.

Initial testing was conducted to verify the sensitivity of the apparatus in sensing increased exhaled temperature when utilizing a mechanical breathing system.

A breathing simulator (Warwick Technology Limited, UK) was used to simulate a human breathing pattern. The simulator comprised an aluminum cylinder and piston driven by a linear actuator and controlled by a computer. The breathing pattern used for the examples had a symmetric sinusoidal pattern at 30 breaths per minute and a minute volume of approximately 30 liters per minute. The breathing simulator outlet fitting was split into two branches, each containing a low pressure drop check valve so that all air exhaled by the breathing machine flowed through one branch and all air inhaled passed through the other branch. The two branches were joined together with a tee which introduced the inhaled and exhaled air flow into the throat of a human headform.

After leaving the breathing machine and before the reaching the tee joining the “inhale” and “exhale” branches air in the “exhale” branch passed through a flow-through electrical heater and a humidifier. The humidifier comprised a 1.5 liter reaction vessel containing heated water. The top of the glass reaction vessel was sealed with a 3-port hemispherical glass cover with the exhaled air entering and leaving the vessel through the two outer ports. The center port of the reaction vessel was used to pass wiring for helical submersible heater inside the vessel to an external controller.

Just before the tee in the “exhale” branch, a heated humidity probe (Vaisala Humicap HMP155, Vaisala Corporation, Helsinki, Finland) and T-type thermocouple probe were inserted into the branch to sense the dewpoint and temperature of exhaled air. A proportional-integral-derivative (PID) controller attached to the humidity probe was used to control the submersible heater in the humidifier to achieve a desired dewpoint of the exhaled air. A PID controller attached to the thermocouple probe was used to control the flow-through heater to achieve a desired temperature of the exhaled air.

The human headform, representing an average sized for a United States worker was constructed with rapid prototyping based on a solid model dataset obtained from the United States National Institute of Occupational Safety and Health. The human headform was constructed with a plastic pipe passing horizontally through the headform from back-to-front and exiting at the mouth. The end of the pipe exiting the back of the headform was attached to the tee connecting the “inhale” and “exhale” branches of the breathing machine.

Temperatures measured at various locations during testing with thermocouples, such as shown in the prototype of FIGS. 7-9. The temperatures were recorded with a computer using National Instruments DAQExpress software at a rate of 90 samples per second. The air temperatures at the mouth of the headform, on the forehead of the headform and next to the exhalation valve of respirators placed on the headform were monitored with thin (0.08 mm diameter) bare junction T-type thermocouples. The temperature of the water in the humidifier was monitored with a stainless steel jacketed T-type thermocouple.

FF400 Breathing Simulator Experiment:

In this experiment, a full face respirator (3M model FF400) with a series of thermocouples and a wireless sensor board capable of tracing the temperature (as well as pressure/humidity) of the in-mask environment such as shown in FIGS. 7-9. A sample breathing trace was graphed. Cyclic noise of the breathing profile was reduced (periods of elevated temperature due to exhalation and periods of reduced temperature due to inhalation) by instead plotting only the peaks and valleys of each breathing profile. Observations that both the thermocouples and the wireless environment sensor track well with the desired output temperature, albeit with a consistent offset. The lag of the wireless sensor was due primarily to its larger thermal mass, however the measurements were very sensitive to changes in environmental temperature. In particular during the transition regions, the Ilus board showed significant thermal gradients in the measured response.

Scott Safety FM Series Breathing Simulator Experiment:

The experiment was on a Scott Safety FM Series full face respirator, using the same thermocouple setup as in the FF400 (measuring at the outlet, the voice diaphragm and in the mask deadzone). For this experiment, equilibrium was established at the temperature 36 C, after which temperature transitions were performed from 36 C->34 C->38 C->36.5 C, using a 32 LPM symmetric sinusoidal breath profile operating at 30 Hz. Peaks and valleys of the oscillating temperature profile were presented based on measurements inside the respirator in these different temperature regimes, highlighting the ability of this measurement modality to track with the underlying temperature of the Warwick system for other mask constructions.

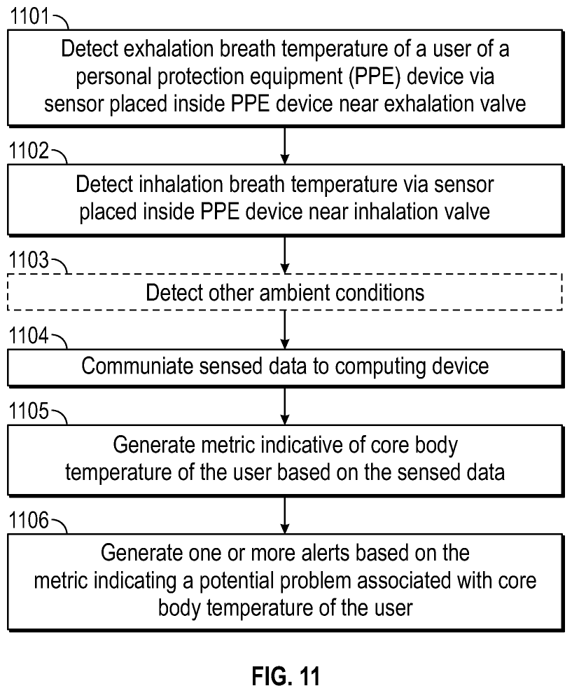

FIGS. 10 and 11 are flow diagrams consistent with techniques of this disclosure. As shown in FIG. 10, a PPE respirator device, such as one of those describe herein, detects exhalation breath temperature of a user (1001). A computing device (either part of the PPE respirator device or external to the PPE respirator device) generates a metric indicative of core body temperature of the user based at least in part on the detected exhalation breath temperature (1002). In some cases, the computing device (or other devices in a connected work environment) may generate one or more alerts based on the metric indicating a potential problem associated with core body temperature of the user (1003).

In some cases, generating one or more alerts (1003) may comprise generating an alert to the user of the PPE respirator device. Alternatively or additionally, generating one or more alerts (1003) may comprise generating an alert to a remote user at a remote location relative to the user of the PPE respirator device. For example, the remote user may be a supervisor or other safety monitor of the user of PPE respirator device. Various different alerts may be generated, depending on what is sensed and depending on the work environment. For example, sensed data may indicate worker distress, or alternatively, it may indicate a need to rest. Other sensed data, such as described above, may be used to assess worker safety or operation of the PPE respirator device, and to identify and generate the proper alert for different situations. In still other cases, the alert may be provided to the user of the PPE respirator device via augmented reality (AR) or mixed reality (MR), particularly if such AR or MR capabilities are incorporated into the PPE respirator device.

In some examples, a computing device that processes sensed data may communicate with other devices over a network, such as by communicating core body temperature of the user or sudden changes or problems with core body temperature. In some cases, a method may include communicating sensed exhale breath temperature data to a local computing device, generating the metric indicative of core body temperature of the user at the local computing device based on the sensed exhale breath temperature data, communicating the metric (or related information) from the local computing device to one or more remote computing devices, generating a local alert at the local computing device in response to the metric indicating a problem with core body temperature, and generating a remote alert at the one or more remote computing devices in response the metric indicating a problem with core body temperature.

In still other examples, a computing device may use exhalation breath temperature alone to detect or identify problems with core body temperature. That is to say, rather than generate a metric, the computing device may simply identify the measured exhalation breath temperature, compare that temperature to one or more threshold or changes relative to a baseline, and generate an alert (or other flag or identifier) if the detected exhalation breath temperature indicates a possible problem with core body temperature. Since exhalation breath temperature generally tracks core body temperature with a fixed offset, it may be easier to merely track exhalation breath temperature relative to thresholds that account for a fixed offset. In this case, the step of generating the metric may be avoided and exhalation breath temperature may be used as a direct proxy for monitoring core body temperature.

FIG. 11 is another flow consistent with techniques of this disclosure. As shown in FIG. 11, a PPE respirator device, such as one of those describe herein, detects exhalation breath temperature of a user (1101) via a sensor placed inside the PPE device near an exhalation valve. In addition, the PPE respirator device detects inhalation breath temperature (1102) via a sensor placed inside the PPE device near an inhalation valve. In some cases, PPE respirator device 1103 may also detect other ambient conditions (1103), either with the breath sensors or with additional sensors. Additional ambient conditions, for example, may include the ambient temperature, or the relative humidity associated with the inhaled air, which can affect core body temperature. The sensors (or a separate communication module connected to the sensors communicates sensed data to a computing device (1104). The computing device (either part of the PPE respirator device or external to the PPE respirator device) generates a metric indicative of core body temperature of the user based at least in part on the sensed data (1105). The computing device (or other devices in a connected work environment) generates one or more alerts based on the metric indicating a potential problem associated with core body temperature of the user of the user (1106).

Again, in some cases, generating one or more alerts (1106) may comprise generating an alert to the user of the PPE respirator device. Alternatively or additionally, generating one or more alerts (1106) may comprise generating an alert to a remote user at a remote location relative to the user of the PPE respirator device. For example, the remote user may be a supervisor or other safety monitor of the user of PPE respirator device. Various different alerts may be generated, depending on what is sensed and depending on the work environment. For example, sensed data may indicate worker distress, or alternatively, it may indicate a need to rest. Other sensed data, such as described above, may be used to assess worker safety or operation of the PPE respirator device, and to identify and generate the proper alert for different situations. In still other cases, the alert may be provided to the user of the PPE respirator device via augmented reality (AR) or mixed reality (MR), particularly if such AR or MR capabilities are incorporated into the PPE respirator device.

The breath sensors may also sense and collect other physiological data, such as breath rate. Also, additional sensors may be used to detect other physiological parameters of the user, or other environmental conditions. This sensed data, like the generated metric indicative of core body temperature, may be analized for any problems or issues that the sensed data may identify in the user. If problems or issues are identified by a computing device, the computing device may generate one or more alerts as described herein.

In some examples, detecting other ambient conditions (1103) may comprise detecting ambient temperature in or around the PPE respirator device, in which case generating the metric (1105) may include generating the metric indicative of core body temperature based at least in part on the detected exhalation breath temperature and the detected ambient temperature. In some examples, generating the metric (1105) may comprise generating the metric indicative of core body temperature based at least in part on the detected exhalation breath temperature and the detected exhalation breath temperature.

In some examples, detecting other ambient conditions (1103) may comprise detecting air humidity inside the PPE respirator device, in which case, generating the metric (1105) may include generating the metric indicative of core body temperature based at least in part on the detected exhalation breath temperature, the detected exhalation breath temperature, and the detected humidity.

In some examples, communicating sensed data (1104) may comprise wireless communicating sensor data from the PPE respirator device to a computing device that is external to the PPE respirator device, wherein the computing device generates the metric.

In some examples, generating the alert (1004 or 1106) may comprises generating the alert in response to the metric indicating core body temperature being over a threshold, i.e. indicating a high core body temperature and possible heat stress. In other examples, generating the alert (1004 or 1106) may comprises generating the alert in response to the metric indicating core body temperature being below a threshold, i.e. indicating a low core body temperature and possible hypothermia in the user.

In some examples, the metric is based on an absolute difference between the detected inhalation breath temperature and the detected exhalation breath temperature. In some example, the computing device may define a baseline based on the detected inhalation breath temperature and the detected exhalation breath temperature during a first time period, in which case generating the one or more alerts (1004 or 1106) may comprise generating the alert in response to the metric changing relative to the baseline during a second time period. The computing device may associate a measured baseline with a given user, and different users may have different baselines due to different physiologies of the different users. As described herein, in still other cases, changes in the metric may be determined based on a time rate of change or based on acceleration in the rate of change of the metric. The metric may be generated in many different ways, and the metric may be analyzed in many different ways to determine whether there is a problem with the core body temperature of the user.

Example 1—A PPE system comprising: a PPE respirator device including a sensor arranged to detect exhalation breath temperature of a user; and a computing device configured to generate a metric indicative of core body temperature of the user based on the detected exhalation breath temperature.

Example 2—The system of example 1, wherein the computing device is part of the PPE respirator device.

Example 3—The system of example 1, wherein the computing device is external to the PPE respirator device.

Example 4—The system of any combination of examples 1-3, wherein the sensor is a first sensor and wherein: the PPE respirator device includes a second sensor arranged to detect ambient temperature; and the computing device is configured to generate the metric based on the detected exhalation breath temperature and the detected ambient temperature.

Example 5—The system of any combination of examples 1-4, wherein the sensor is a first sensor and wherein: the PPE respirator device includes a second sensor arranged to detect inhalation breath temperature; and the computing device is configured to generate the metric based on the detected exhalation breath temperature and the detected inhalation breath temperature.

Example 6—The system of any combination of examples 1-5, wherein the sensor is a first sensor and wherein: the PPE respirator device includes one or more second sensors arranged to detect inhalation breath temperature and humidity; and the computing device is configured to generate the metric based on the detected exhalation breath temperature, the detected inhalation breath temperature, and the detected humidity.

Example 7—The system of any combination of examples 1-6, wherein PPE respirator device includes a wireless communication module connected to the sensor and wherein the wireless communication module wireless communicates sensor data to the computing device.

Example 8—The system of any combination of examples 1-7, wherein computing device is configured to generate an alert in response to the metric indicating a potential problem related to core body temperature.

Example 9—The system of any combination of examples 1-8, wherein the computing device is configured to generate the alert based on the metric indicating core body temperature being over a threshold.

Example 10—The system of any combination of examples 1-9, wherein the computing device is configured to generate the alert based on the metric indicating core body temperature being below a threshold.

Example 11—The system of any combination of examples 1-10, wherein the metric is based on an absolute difference between the detected inhalation breath temperature and the detected exhalation breath temperature, and wherein the computing device is configured to generate an alert in response to the metric indicating a potential problem related to core body temperature.

Example 12—The system of any combination of examples 1-11, wherein the computing device is configured to define a baseline of the user based on the detected inhalation breath temperature and the detected exhalation breath temperature during a first time period and to generate the alert in response to the metric changing relative to the baseline in a second time period.

Example 13—The system of any combination of examples 1-12, wherein the computing device is associated with the user of the PPE respirator device.

Example 14—The system of any combination of examples 1-13, wherein the computing device is associated with a supervisor of the user of the PPE respirator device.

Example 15—The system of any combination of examples 1-14, wherein the PPE respirator device comprises one or more of: a full-face respirator device; a half-face respirator device; a negative pressure reusable respirator system; a powered air purifying respirator (PAPR); a filtered face piece respirator (FFR); a respirator that forms part of a self-contained breathing apparatus (SCBA); a respirator that forms part of a safety helmet or mask; a respirator that forms part of firefighter safety equipment; a respirator that forms part of military equipment; and/or a medical respirator device.

Example 16—The system of any combination of examples 1-15, wherein PPE respirator device comprises a facepiece designed to fit over a nose and a mouth of a user, the facepiece defining a sealable space formed around the nose and the mouth of the user for delivering air to the user, wherein the facepiece includes an exhalation valve and wherein the sensor is positioned proximate to the exhalation valve.

Example 17—The system of any combination of examples 1-16, wherein the sensor is positioned inside the sealable space proximate to the exhalation valve of the facepiece of the PPE respirator device.

Example 18—The system of any combination of examples 1-17, wherein the sensor is positioned between the mouth of the user and the exhalation valve of the PPE respirator device.

Example 19—The system of any combination of examples 1-18, wherein PPE respirator device comprises a facepiece designed to fit over a nose and a mouth of a user, wherein the facepiece includes an inhalation valve and an exhalation valve, wherein the first sensor is positioned proximate to the exhalation valve of the facepiece, and wherein the second sensor is positioned proximate to the inhalation value of the facepiece.

Example 20—The system of any combination of examples 1-19, wherein the first sensor is positioned inside the sealable space proximate to the exhalation valve of the facepiece and wherein the second sensor is positioned inside the sealable space proximate to the inhalation valve of the facepiece.

Example 21—The system of any combination of examples 1-20, wherein the PPE respirator device includes a wireless communication module connected to the sensor and wherein the wireless communication module wireless communicates sensor data to the computing device, wherein the wireless communication module is positioned inside a vented cap that covers the inhalation valve and the exhalation valve of the facepiece.

Example 22—A PPE respirator device comprising: a facepiece designed to fit over a nose and a mouth of a user, the facepiece defining a sealable space formed around the nose and the mouth of the user for delivering air to the user, wherein the facepiece includes an inhalation valve and an exhalation valve, a first sensor positioned proximate to the exhalation valve and configured to detect exhalation breath temperature, and a second sensor positioned proximate to the inhalation valve and configured to detect inhalation breath temperature.

Example 23—The PPE respirator device of example 22, wherein the PPE respirator device includes a wireless communication module connected to the first sensor and the second sensor and wherein the wireless communication module wireless communicates sensor data to a computing device.

Example 24—The PPE respirator device of example 23, wherein the wireless communication module is positioned inside a vented cap that covers the inhalation valve and the exhalation valve of the facepiece.

Example 25—The PPE respirator device of any combination of examples 22-24, wherein the first sensor is positioned inside the sealable space proximate to the exhalation valve of the facepiece and wherein the second sensor is positioned inside the sealable space proximate to the inhalation valve of the facepiece.

Example 26—The PPE respirator device of any combination of examples 22-25, wherein the first sensor is positioned outside the sealable space proximate to the exhalation valve of the facepiece and wherein the second sensor is positioned outside the sealable space proximate to the inhalation valve of the facepiece.

Example 27—The PPE respirator device of any combination of examples 22-25, wherein the first sensor is positioned inside the sealable space proximate to the exhalation valve of the facepiece and wherein the second sensor is positioned outside the sealable space proximate to the inhalation valve of the facepiece.

Example 28—A method comprising: detecting exhalation breath temperature inside a PPE respirator device; and generating a metric indicative of core body temperature based at least in part on the detected exhalation breath temperature.

Example 29—The method of example 28, further comprising: detecting ambient temperature in or around the PPE respirator device; and generating the metric indicative of core body temperature based at least in part on the detected exhalation breath temperature and the detected ambient temperature.

Example 30—The method of example 28 or 28, further comprising: detecting inhalation breath temperature inside the PPE respirator device; and generating the metric indicative of core body temperature based at least in part on the detected exhalation breath temperature and the detected inhalation breath temperature.

Example 31—The method of any combination of examples 28-30, further comprising: detecting inhalation breath temperature and air humidity inside the PPE respirator device; and generating the metric indicative of core body temperature based at least in part on the detected exhalation breath temperature, the detected inhalation breath temperature, and the detected humidity.

Example 32—The method of any combination of examples 28-31, further comprising: wirelessly communicating sensor data from the PPE respirator device to a computing device that is external to the PPE respirator device, wherein the computing device generates the metric.

Example 33—The method of any combination of examples 28-32, further comprising: generating an alert in response to the metric indicating a potential problem related to core body temperature.

Example 34—The method of any combination of examples 28-33, wherein generating the alert comprises generating the alert in response to the metric indicating core body temperature being over a threshold.

Example 35—The method of any combination of examples 28-34, wherein generating the alert comprises generating the alert in response to the metric indicating core body temperature being below a threshold.

Example 36—The method of any combination of examples 28-32, wherein the metric is based on an absolute difference between the detected inhalation breath temperature and the detected exhalation breath temperature, the method further comprising: generating an alert in response to the metric indicating a potential problem related to core body temperature.

Example 37—The method of any combination of examples 28-36, the method further comprising: defining a baseline based on the detected inhalation breath temperature and the detected exhalation breath temperature during a first time period; and generating the alert in response to the metric changing relative to the baseline during a second time period.

Example 38—The method of any combination of examples 28-37, further comprising associating the defined baseline with the user.

Example 39—The method of any combination of examples 28-38, wherein the PPE respirator device comprises one or more of: a full-face respirator device; a half-face respirator device; a negative pressure reusable respirator system; a powered air purifying respirator (PAPR); a filtered face piece respirator (FFR); a respirator that forms part of a self-contained breathing apparatus (SCBA); a respirator that forms part of a safety helmet or mask; a respirator that forms part of firefighter safety equipment; a respirator that forms part of military equipment; and/or a medical respirator device.

Example 40—A PPE system comprising: a PPE respirator device including a sensor arranged to detect exhalation breath temperature of a user; and a computing device configured to generate one or more alerts in response to the detected exhalation breath temperature indicating a potential problem with the user.

Example 41—The PPE system of claim 40 in combination with features set forth in any of examples 1-21.

Although the devices, methods and systems of the present disclosure have been described with reference to specific exemplary embodiments, those of ordinary skill in the art will readily appreciate that changes and modifications may be made thereto without departing from the spirit and scope of the present disclosure.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

1. A personal protection equipment (PPE) system comprising:
a PPE respirator device including a sensor arranged to detect exhalation breath temperature of a user; and
a computing device configured to generate a metric indicative of core body temperature of the user based on the detected exhalation breath temperature.

2. The system of claim 1, wherein the computing device is part of the PPE respirator device.

3. The system of claim 1, wherein the computing device is external to the PPE respirator device.

4. The system of claim 1, wherein the sensor is a first sensor and wherein:
the PPE respirator device includes a second sensor arranged to detect ambient temperature; and
the computing device is configured to generate the metric based on the detected exhalation breath temperature and the detected ambient temperature.

5. The system of claim 1, wherein the sensor is a first sensor and wherein:
the PPE respirator device includes a second sensor arranged to detect inhalation breath temperature; and
the computing device is configured to generate the metric based on the detected exhalation breath temperature and the detected inhalation breath temperature.

6. The system of claim 1, wherein the sensor is a first sensor and wherein:
the PPE respirator device includes one or more second sensors arranged to detect inhalation breath temperature and humidity; and
the computing device is configured to generate the metric based on the detected exhalation breath temperature, the detected inhalation breath temperature, and the detected humidity.

7. The system of claim 3, wherein PPE respirator device includes a wireless communication module connected to the sensor and wherein the wireless communication module wireless communicates sensor data to the computing device.

8. The system of claim 1, wherein computing device is configured to generate an alert in response to the metric indicating a potential problem related to core body temperature.

9. The system of claim 8, wherein the computing device is configured to generate the alert based on the metric indicating core body temperature being over a threshold.

10. The system of claim 8, wherein the computing device is configured to generate the alert based on the metric indicating core body temperature being below a threshold.

11. The system of claim 5, wherein the metric is based on an absolute difference between the detected inhalation breath temperature and the detected exhalation breath temperature, and wherein the computing device is configured to generate an alert in response to the metric indicating a potential problem related to core body temperature.

12. The system of claim 11, wherein the computing device is configured to define a baseline of the user based on the detected inhalation breath temperature and the detected exhalation breath temperature during a first time period and to generate the alert in response to the metric changing relative to the baseline in a second time period.

13. The system of claim 1, wherein the computing device is associated with the user of the PPE respirator device.

14. The system of claim 1, wherein the computing device is associated with a supervisor of the user of the PPE respirator device.

15. The system of claim 1, wherein the PPE respirator device comprises one or more of:
a full-face respirator device;
a half-face respirator device;
a negative pressure reusable respirator system;
a powered air purifying respirator (PAPR);
a filtered face piece respirator (FFR);
a respirator that forms part of a self-contained breathing apparatus (SCBA);
a respirator that forms part of a safety helmet or mask;
a respirator that forms part of firefighter safety equipment;
a respirator that forms part of military equipment; and/or
a medical respirator device.

16. The system of claim 1, wherein PPE respirator device comprises a facepiece designed to fit over a nose and a mouth of a user, the facepiece defining a sealable space formed around the nose and the mouth of the user for delivering air to the user, wherein the facepiece includes an exhalation valve and wherein the sensor is positioned proximate to the exhalation valve.

17-21. (canceled)

22. A personal protection equipment (PPE) respirator device comprising:
a facepiece designed to fit over a nose and a mouth of a user, the facepiece defining a sealable space formed around the nose and the mouth of the user for delivering air to the user, wherein the facepiece includes an inhalation valve and an exhalation valve,
a first sensor positioned proximate to the exhalation valve and configured to detect exhalation breath temperature; and
a second sensor positioned proximate to the inhalation valve and configured to detect inhalation breath temperature.

23. The PPE respirator device of claim 22, wherein the PPE respirator device includes a wireless communication module connected to the first sensor and the second sensor and wherein the wireless communication module wireless communicates sensor data to a computing device.

24. The PPE respirator device of claim 23, wherein the wireless communication module is positioned inside a vented cap that covers the inhalation valve and the exhalation valve of the facepiece.

25. The PPE respirator device of claim 22, wherein the first sensor is positioned inside the sealable space proximate to the exhalation valve of the facepiece and wherein the second sensor is positioned inside the sealable space proximate to the inhalation valve of the facepiece.

26-40. (canceled)
